(19) United States
(12) Patent Application Publication
(10) Pub. No.: US 2026/0337569 A1
(43) Pub. Date: Oct. 29, 2026

(54) Identification of a Common Precursor to Effector and Regulatory Tissue Imprinted CD4+ T Cells and Therapeutic Use Thereof

(71) Applicants: Institut Curie, Paris (FR); Institut National de la Santé et de la Recherche Médicale, Paris (FR)

(72) Inventors: Eliane Piaggio, Paris (FR); Jimena Tosello, Paris (FR); Christine Sedlik, Le Plessis Bouchard (FR); Wilfrid Richer, Rueil Malmaison (FR); JOSHUA WATERFALL, PARIS (FR)

(73) Assignees: Institut Curie, Paris (FR); Institut National de la Santé et de la Recherche Médicale, Paris (FR)

(21) Appl. No.: 19/109,108

(22) PCT Filed: Sep. 6, 2023
(86) PCT No.: PCT/EP2023/074509

§ 371 Date: Mar. 5, 2025

(30) Foreign Application Priority Data
Sep. 6, 2022 (EP) 22306321.5
Nov. 17, 2022 (EP) 22306699.4

(51) Int. Cl. C12N 5/0783; A61K 40/11; A61K 40/32; A61P 35/00; C07K 14/705

(52) U.S. Cl. CPC C12N 5/0637; A61K 40/11; A61K 40/32; A61P 35/00; C07K 14/7051; C12N 2510/00

(57) ABSTRACT

The invention relates to tissue-imprinting molecular program and the associated T cell progenitors in tumors and draining lymph nodes and their therapeutic application.

### FIELD OF THE INVENTION

The invention pertains to the field of immunotherapy, in particular of cancer. The invention relates to tissue-imprinting molecular program and the associated T cell progenitors in tumors and draining lymph nodes and their therapeutic application.

### BACKGROUND OF THE INVENTION

Conventional CD4+ T cells play a multifaceted role in cancer immunology by directly destroying tumor cells, and by indirectly supporting the effector function and differentiation of CD8+ T cells and B cells1. Conversely, regulatory CD4+ T cells (Tregs) contribute to the development of cancer by imposing an immunosuppressive microenvironment and eventually killing tumor-specific T cells². Thus, CD4+ T cells constitute a plethora of different subsets that regulate the balance between inflammatory and tolerogenic immune responses, locally in the tumor and in the draining lymph nodes (LNs), where the adaptive immune response initiates³.

The advent of high throughput single cell (sc) technologies and the development of powerful bioinformatics tools to integrate these data, have provided unprecedented analytical tools to study the characteristics of T cells in cancer patients, yielding the first atlases of CD4+ T cells in the tumor microenvironment⁴⁻⁶. Data on human Tregs are scarcer⁷⁻⁹, and comprehensive studies of CD4+ T cells in tumor draining LNs and their relationship with the cells in the tumor is missing¹⁰. Tumor-draining lymph nodes (LNs) are the most important sites for the priming of immune responses, yet their role in the orchestration of the anti-tumor CD4+ T cell response remains little explored. It is then timely to use these novel technologies to revisit fundamental unanswered questions about CD4+ T cell priming, activation, differentiation, migration, and tissue adaptation in the context of cancer.

### SUMMARY OF THE INVENTION

To address these issues, the inventors performed coupled scRNA-seq/scTCR-seq and scATAC-seq on sorted CD4+ Tconvs and Tregs from blood, LNs and tumors from treatment-naïve NSCLC patients, and used the TCR as lineage barcodes to track cell fates. Here, the inventors present an integrative single cell analysis of transcriptome, T cell receptor (TCR), and chromatin accessibility profiles of CD4+ conventional (Tconv) and regulatory (Treg) T cells from matched blood, LNs and tumors of treatment naïve Non-Small Cell Lung Cancer (NSCLC) patients. Among multiple Tconv and Treg subpopulations, the inventors identify a subgroup of TCR-activated and tissue-imprinted cells sharing a common regulatory program governed by JUN and BATF. Using the TCR as a lineage barcode, the inventors find that tumor-expanded Treg and Tconv clones, luckily representing tumor-specific cells, mostly present a tissue-imprinted phenotype and are also present in the LNs. Moreover, the inventors map tumor-expanded Treg and Tconv migration and characterize their LNs and tumors associated features. Additionally, using trajectory analysis, the inventors identify Treg-follicular like cells (Treg-FL) in the LNs as precursors of tissue-imprinted effector Tregs and Tconvs, which are enriched in signatures of tumor specificities. Finally, the combined multiomics analysis of cancer patient samples revealed the transcriptional program underlying the developmental bifurcation of precursor Treg-FLs into tissue-imprinted Treg or Tconv cells, which could guide the design of novel immune modulatory therapies. These results elucidate the transcriptomic and epigenetic profile of different human tumor-associated CD4+ T cell subpopulations in the three tissues, illuminating the LN-occurring processes that orchestrate the anti-tumor CD4+ T cell response, and providing novel cues for the therapeutic manipulation of these responses. In particular, the identified tissue-imprinted T cells enriched in tumor specificities, as well as their common precursor represent promising targets for therapeutic modulation.

Therefore, a first aspect if the invention relates to a human T cell precursor having a phenotype characterized by the expression of the markers CD3 and CD4; the expression of the marker Forkhead Box P3 (FOXP3) and/or high expression of the marker Interleukin 2 Receptor Subunit Alpha (IL2RA); the expression or high expression, of the marker Inducible T Cell Costimulator (ICOS); and the high expression of the marker Programmed Cell Death 1 (PD1).

In some embodiments, the T cell precursor further comprises high expression of at least one marker selected from the group consisting of: Cytotoxic T-Lymphocyte Associated Protein 4 (CTLA-4), Basic Leucine Zipper ATF-Like Transcription Factor (BATF), Islet Cell Autoantigen 1 (ICA1), Cochlin (COCH), Pro-Melanin Concentrating Hormone (PMCH), Zinc Finger Protein 281 (ZNF281), Thy-1 Cell Surface Antigen (THY.1), CD200, Interferon Regulatory Factor 4 (IRF4), T Cell Immunoreceptor With Ig And ITIM Domains (TIGIT), Thymocyte Selection Associated High Mobility Group Box (TOX), B-Lymphocyte-Induced Maturation Protein 1 (PRDM1), C-X-C Motif Chemokine Ligand 13 (CXCL13), B-and T-lymphocyte attenuator (BTLA), Insulin-like growth factor 1 receptor (CD221), Glucocorticoid-induced TNFR-related protein (TNFRSF18), Integrin Subunit Alpha 4 (ITGA4), Interleukin 21 Receptor (IL21R), leucine rich repeat containing 32 (LRRC32), Rhotekin 2 (RTKN2), IKAROS Family Zinc Finger 2 (IKZF2), and CD151.

In some particular embodiments, the human T cell precursor comprises expression of the markers CD3, CD4 and ICOS; and high expression of the markers IL2RA, CD200, BTLA and PD1. In some other particular embodiments, the human T cell precursor comprises expression of the markers CD3, CD4, FOXP3 and ICOS; and high expression of the markers CD200, BTLA and PD1. Preferably, the human T cell precursor comprises expression of the markers CD3, CD4 and ICOS; and high expression of the markers IL2RA, CD200, BTLA and PD1. In some embodiments, the T cell precursor is a precursor of both regulatory T cells (Tregs) and effector conventional T cells (Tconvs).

In some embodiments, the T cell precursor or derived regulatory T cells or conventional T cells express tissue-imprinting markers. In particular embodiments, the T cell precursor or derived regulatory T cells or conventional T cells express at least one marker of tissue residency chosen from C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1 (ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT; and do not express at least one marker of blood circulation chosen from C-C Motif Chemokine Receptor 7 (CCR7), Selectin L (SELL), Sphingosine-1-phosphate receptor 1 (S1PR1) and Krüppel-like Factor 2 (KLF2). In some preferred embodiments, the derived regulatory T cells or conventional T cells further express at least one transcription factor of T cell activation and differentiation chosen from Jun Proto-Oncogene, AP-1 Transcription Factor Subunit (JUN), RELB Proto-Oncogene, NF-KB Subunit (RELB), Fos Proto-Oncogene, AP-1 Transcription Factor Subunit (FOS), Interferon regulatory factor 4 (IRF4), and Basic Leucine Zipper ATF-Like Transcription Factor (BATF), in particular further expressing at least BATF; are mainly found in both lymph nodes and tumors, but not in blood; and are enriched in tumor reactivity.

In some embodiments, the T cell precursor is isolated from blood, tonsil, spleen, bone marrow, lymph node or tumor; preferably tumor and/or tumor-draining lymph node.

In some embodiments, the T cell precursor is enriched in lymph node and tumor compared to blood.

In some embodiments, the T cell precursor is produced from induced pluripotent stem cells (iPS).

Another aspect of the invention relates to a method of differentiation of the precursor cell according to the present disclosure into regulatory T cells, comprising inducing the expression of a transcription factor program comprising upregulation of at least one of MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1,IRF1,ID3, NFKB2, NFKB1, HIVEP3, HMGA1, YY1, FLI1 and ATF3; and downregulation of at least one of RBPJ, FOXN2, REST, ZNF75A, FOXO3, KLF12, NFATC2, MAF, RUNX2, NR3C1, ZNF281, HIVEP1, HIVEP2, LEF1 and TCF7 in the cell. In some preferred embodiments, the transcription factor program comprises upregulation of at least one of: MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3 and HMGA1; and downregulation of at least one of RBPJ, FOXN2, REST, ZNF75A, FOXO3, KLF12, NFATC2, MAF, RUNX2 and NR3C1 in the cell. In some more preferred embodiments, the transcription factor program comprises upregulation of at least one of: IRF1, ID3, NFKB2, NFKB1, HIVEP3 and HMGA1; and downregulation of at least one of NFATC2, MAF, RUNX2 and NR3C1 in the cell.

Another aspect of the invention relates to a method of differentiation of the precursor cell according to the present disclosure into conventional T cells, comprising inducing the expression of a transcription factor program comprising upregulation of at least one of RORA, PPARG, CREM, NR2C2, ETS1, SP4, CEBPZ, BCL11B, ZNF75D, NFATC2, MAF, RUNX2, NR3C1, YY1, FLI1 and ATF3; and downregulation of at least one of RELB, ZEB1, TOPORS, ZBTB7A, IRF1, ID3, NFKB2, NFKB1, HIVEP3, HMGA1, ZNF281, HIVEP1, HIVEP2, LEF1 and TCF7 in the cell. In some preferred embodiments, the transcription factor program comprises upregulation of at least one of RORA, PPARG, CREM, NR2C2, ETS1, SP4, CEBPZ, BCL11B, ZNF75D, NFATC2, MAF, RUNX2 and NR3C1; and downregulation of at least one of RELB, ZEB1, TOPORS, ZBTB7A, IRF1, ID3, NFKB2, NFKB1, HIVEP3 and HMGA1 in the cell. In some more preferred embodiments, the transcription factor program comprises upregulation of at least one of NFATC2, MAF, RUNX2 and NR3C1; and downregulation of at least one of IRF1, ID3, NFKB2, NFKB1, HIVEP3 and HMGA1 in the cell.

In some embodiments of the methods of differentiation according to the invention, expression of the transcription factor program is induced using modulator(s) of the transcription factor(s) expression or activity selected from the group consisting of: small organic molecules, antibodies, peptides, aptamers, interfering RNA molecules, antisense nucleic acids, ribozymes, genome and epigenome editing complexes, dominant negative mutants, protein fragments and other agonists or antagonists.

In some embodiments of the methods according to the invention, the modulation of transcription factors involved in the differentiation may alter the stability, phenotype, and or tissue retention properties of blood derived Tregs and Tconvs.

In some embodiments, the methods of differentiation according to the invention, further comprise the expansion of the derived regulatory T cells or conventional T cells.

The invention also relates to a screening method for inducers of differentiation of a T cell precursor according to the present disclosure, comprising: (a) administering a modulator of the expression or activity of a transcription factor according to the present disclosure to a T cell precursor according to the present disclosure, (b) measuring the level of expression of the transcription factor in the T cell precursor, and (c) identifying a modulator that upregulates a transcription factor that is upregulated in the transcription factor program according to the present disclosure or downregulates a transcription factor that is downregulated in the transcription factor program according to the present disclosure, in the treated precursor cell as compared to untreated precursor cell.

The invention provides a modified immune cell obtained from the T cell precursor according to the present disclosure, or the derived regulatory T cells or conventional T cells according to the present disclosure. In some preferred embodiments, the modified immune is genetically engineered to express a chimeric antigen receptor or exogenous TCR specific for a target antigen.

The invention also provides a CAR-T cell or TCR-T cell which is modified to stimulate expression of at least one marker of tissue residency chosen from C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1 (ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT; in particular chosen from C-X-C Motif Chemokine Receptor 3 (CXCR3), C-X-C Motif Chemokine Receptor 6 (CXCR6), CD69, Interleukin 1 Receptor Like 1 (IL-1RL1) and B-Lymphocyte-Induced Maturation Protein 1 (PRDM1), and/or inhibit expression of at least one marker of blood circulation chosen from C-C Motif Chemokine Receptor 7 (CCR7), Selectin L (SELL), Sphingosine-1-phosphate receptor 1 (S1PR1) and Krüppel-like Factor 2 (KLF2).

The invention further provides a TCR-T cell which comprises an engineered TCR from regulatory T cells or conventional T cells according to the present disclosure.

A further aspect of the invention relates to molecular signature of migrating or resident regulatory T cells or conventional T cells in the tumor or lymph node selected from the group consisting of:
A signature of migrating regulatory T cells Tregs present in the lymph node comprising the expression of CCR8, HAVCR2, IL2RB, and LAIR2 genes;
A signature of migrating conventional T cells present in the the lymph node comprising the expression of GLUL, HLA-A, HLA-DRA, NKG7 and SEC61G genes;
A signature of migrating regulatory T cells present in the Tumor comprising the expression of: CCR8, CTLA4 and SDC4 genes;
A signature of migrating conventional T cells present in the tumor comprising the expression of: CLEC2B, and HLA-DQA1 genes;
A signature of resident regulatory T cells present in the lymph node comprising the expression of: CCR7 and LEF1 genes;.
A signature of resident conventional T cells present in the lymph node are comprising the expression of: CCR7, LEF1, SELL, SESN1 and TCF7 genes;
A signature of resident regulatory T cells present in the Tumor comprising the expression of: ICAM3, IL12RB2, IL27RA, LAMP1, LGALS3, TNFRSF14, TNFRSF18 and TNFRSF4 genes; and
A signature of resident conventional T cells present in the tumor comprising the expression of: CALM1, CALM3, ICAM3, IL6ST, LGALS3, SIRPG, BTLA and CXCL13 genes.

The invention also relates to a modulator of the molecular signature according to the present disclosure for use in the treatment of cancer to modulate immune infiltration in a tumor by favoring the tissue homing and/or migration in the tumor of effector conventional T cells and/or CD8+ T cells, or impairing the tissue homing and/or migration in the tumor of regulatory T cells.

The invention further provides a pharmaceutical composition comprising a therapeutically effective amount of T cell precursor according to the present disclosure, derived regulatory T cells or conventional T cells according to the present disclosure or modified immune cell according to the present disclosure; CAR-T cell or TCR-T cell according to the present disclosure or inducer of differentiation of said T cell precursor, regulatory of conventional T cells.

In some embodiments of the pharmaceutical composition, the T cell is autologous or HLA-compatible.

The invention also relates to a pharmaceutical composition according to the present disclosure for use in the treatment of cancer, acute or chronic inflammatory diseases, autoinflammatory diseases, miscarriage, allergic diseases, autoimmune or infectious diseases, graft-versus-host disease and graft-rejection, and tissue repair.

### DETAILED DESCRIPTION OF THE INVENTION

The invention relates to a human T cell precursor, to the regulatory T cells and conventional T cells differentiated from the precursor, in particular imprinted T cells, and their application for the treatment of various diseases such as cancer, infectious and immune diseases. The invention encompasses signatures of migration and tissue-imprinting (or tissue-residency) of regulatory T cells and conventional T cells and their therapeutic applications.

### Definitions

A used herein, “precursor(s)”, “precursor cell(s)”, “progenitor(s)”, “progenitor cell(s)” refers to a cell(s) with pluripotential capacity (i.e. pluripotent cell(s)). A pluripotent cell has the capacity to differentiate into a plurality (at least two) of functionally specialized cells of one type of tissue or different types of tissue in the body. The precursor cell according to the invention is a human cell. The precursor cell according to the invention is a precursor of human T cells (i.e., human T cell precursor). A precursor cell according to the invention refers to an isolated precursor cell.

As used herein, T cells refer to CD3+cells; “regulatory T cells”, “T regulatory cells”, “Tregs”, “Treg” or “Treg cells” refer to CD3+CD4+Foxp3+ CD25high cells; CD4+ Foxp3+ T cells; CD4+ CD25high T cells (in particular for the isolation without intracellular staining); or CD4+, CD25+ and CD127-T cells and “T conventional cells”, “Tconv cells”, “Tconvs”or “Tconv” refer to CD3+CD4+Foxp3-CD25low cells (or CD4+Foxp3-T cells). Tregs and Tconv are both CD4+ T cells. Tconv are effector T cells. T cells as used herein refers to functional T cells.

The term “marker” as used herein means “molecular marker” or “molecular signature” and refers to a specific gene or gene product (RNA or protein). A marker is in particular a cell marker that may be a cell-surface or intracellular marker. A marker includes any one of the markers disclosed herein such as any marker disclosed in the examples or figures of the present disclosure.

As used herein, «gene signature » or «gene expression signature » refers to a single or combined group of genes in a cell with a uniquely characteristic pattern of gene expression or peak accessibility specific for a unique characteristic feature of the cell such as for example cell differentiation, tissue-imprinting, migration, tissue-residency and others.

As used herein, “expression” of a marker in a cell refers to a detectable level of the marker in the cell irrespective of its expression level. “high expression”, “high expression level” “overexpression” of a marker in a cell refers to a high level of expression of the marker in the cell. The level of expression of markers in cells can be measured by standard quantitative or semi-quantitative techniques that are well-known in the art, and for example disclosed herein. Expression of a marker refers to gene and/or protein expression of the marker, in particular gene and protein expression of the marker.

As used herein, a cell expressing tissue-imprinting markers refers to a cell such as CD4+ T cell: (i) expressing markers of tissue residency such as C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1) and others as disclosed in the examples or figures of the present disclosure; in particular expressing markers of tissue residency such as C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1(ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT and (ii) not expressing markers of blood circulation, such as C-C Motif Chemokine Receptor 7 (CCR7), Selectin L (SELL), Sphingosine-1-phosphate receptor 1 (S1PR1), Krüppel-like Factor 2 (KLF2), and others as disclosed in the examples or figures of the present disclosure. The cell may express one or more or all of the of tissue residency markers listed above or may not express one or more or all of the markers of blood circulation listed below.

As used herein, “tissue-imprinted cells” or “tissue-imprinted CD4+ T cells” refer to a group of T conventional and regulatory cells enriched in tumor reactivity and expressing a tissue-imprinted program. “tissue imprinted CD4+ T cells” are characterized as having the following distinguishing features: i) they express Basic Leucine Zipper ATF-Like Transcription Factor (BATF), a key transcription factor (TF) determining the molecular program tissue residency described in the literature for Tregs and CD8+ memory cells; ii) they express molecules associated to tissue residency, such as CXCR3, CXCR6, CD69, IL1RL1 and PRDM1; in particular molecules associated to tissue residency such as C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1 (ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT; (iii) they do not express known determinants of T cell circulation and tissue egress such as CCR7, SELL, S1PR1, and KLF2; and (iv) they are mainly found in both LNs and tumors, but not in blood.

As used herein, T cells “enriched in tumor reactivity” refer to T cells enriched with clonally expanded TCR in the tumor and/or expression of molecular features related with tumor specific cells as described in the literature (Lowery, F. J. et al., Science 375, 877-884 (2022)).

The different types and subtypes of T cells, in particular CD4+ T cells, as disclosed herein can be analyzed and sorted by routine techniques known in the art such as flow cytometry assisted cell sorting or magnetic cell separation, using appropriate antibodies as disclosed in the examples. Flow cytometry such as flow cytometry assisted cell sorting can be used to isolate the T cell precursor according to the present disclosure. Flow cytometry can also use to determine the expression levels of cell-surface and intracellular markers in the T cell precursor according to the present disclosure.

A modulator may be an activator or an inhibitor. A modulator may inhibit or stimulate expression of the target (gene) or activity or the target (gene product, in particular protein). The target is any gene of interest such as any molecular marker or transcription factor as disclosed herein. As used herein, “inhibiting or stimulating” the expression or activity of said target includes a direct or indirect inhibition or stimulation. A direct inhibition or stimulation is directed specifically to the target. An indirect inhibition or stimulation is directed to any effector of the target biological or signalling pathway such as with no limitations: a ligand or co-ligand, a receptor or co-receptor of said target; a co-factor or a co-effector of said target biological or signalling pathway.

As used herein, the term “cancer” refers to any member of a class of diseases or disorders characterized by uncontrolled division of cells and the ability of these cells to invade other tissues, either by direct growth into adjacent tissue through invasion or by implantation into distant sites by metastasis. Metastasis is defined as the stage in which cancer cells are transported through the bloodstream or lymphatic system. The term cancer according to the present invention also comprises cancer metastases and relapse of cancer. Cancers are classified by the type of cell that the tumor resembles and, therefore, the tissue presumed to be the origin of the tumor. For example, carcinomas are malignant tumors derived from epithelial cells. This group represents the most common cancers, including the common forms of breast, prostate, lung, and colon cancer. Lymphomas and leukemias include malignant tumors derived from blood and bone marrow cells. Sarcomas are malignant tumors derived from connective tissue or mesenchymal cells. Mesotheliomas are tumors derived from the mesothelial cells lining the peritoneum and the pleura. Gliomas are tumors derived from glia, the most common type of brain cell. Germinomas are tumors derived from germ cells, normally found in the testicle and ovary. Choriocarcinomas are malignant tumors derived from the placenta. As used herein, “cancer” refers to any cancer type including solid and liquid tumors.

As used herein infectious diseases refers to any disease caused by a pathogenic agent or microorganism such as virus, bacteria, fungi, parasite and the like.

As used herein, the term “subject” or “individual” refers to a human. The subject may or may not be affected by a disease. A “patient” refers to a subject affected by a disease.

The term “treating” or “treatment”, as used herein, means reversing, alleviating, inhibiting the progress of, or preventing the disorder or condition to which such term applies, or reversing, alleviating, inhibiting the progress of, or preventing one or more symptoms of the disorder or condition to which such term applies. As used herein, the terms “treatment” or “treat” refer to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and include suppression of clinical relapse. The treatment may be administered to a patient having a medical disorder or who ultimately may acquire the disorder, in order to prevent, cure, delay the onset of, reduce the severity of, or ameliorate one or more symptoms of a disorder or recurring disorder, or in order to prolong the survival of a patient beyond that expected in the absence of such treatment.

“Treating cancer” includes, without limitation, reducing the number of cancer cells or the size of a tumor in the patient, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms, slowing the progression of symptoms and others.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

### T Cell Precursor and Derived Tconvs and Tregs

One aspect of the invention relates to a human T cell precursor having a phenotype characterized by the expression of the markers CD3 and CD4; the expression of the marker FOXP3 (Forkhead Box P3) and/or high expression of the marker IL2RA (Interleukin 2 Receptor Subunit Alpha or CD25); the expression or high expression, of the marker ICOS (Inducible T Cell Costimulator) and the high expression of the marker PD1 (Programmed Cell Death 1).

Since CD3 is a marker specific for T cells, the human T cell precursor according to the invention may be defined as a subset or population of CD4+ T cells having a phenotype characterized by the expression of the marker FOXP3 (Forkhead Box P3) and/or high expression of the marker IL2RA (Interleukin 2 Receptor Subunit Alpha or CD25); the expression or high expression, of the marker ICOS (Inducible T Cell Costimulator) and the high expression of the marker PD1 (Programmed Cell Death 1).

The precursor cell according to the invention may be isolated from various human samples comprising T cells that are well-known in the art. Such samples include in particular blood, lymphoid organ, and/or tumor sample. Lymphoid organ may be for example tonsil, spleen, bone marrow, and/or lymph node. Lymph node includes tumor draining lymph node. As used herein, “tumor” includes tumor tissue and tumor fluid. Tumor tissue includes primary tumor, metastasis and tumor draining lymph node, in particular metastatic tumor draining lymph node. Tumor fluid includes all fluids draining the tumor. An example of tumor sample is a tumor biopsy.

In some particular embodiments, the precursor cell is isolated from tumor and/or tumor-draining lymph node sample. In some particular embodiments, precursor cell is enriched in lymph node and tumor compared to blood.

The precursor cell can be isolated using standard T cell isolation techniques that are well-known in the art and disclosed in the examples of the present application, such as magnetic enrichment and others. The methods usually comprise a tissue processing step before selecting for the precursor cells in the sample. The precursor cells can be selected with any combinations of the markers set forth herein. Selection can be performed by routine techniques in art, such as by FACS analysis and cell sorting such as magnetic cell-sorting using antibodies specific for the markers, for example, as described in the Examples. In particular, T cells may be identified as negative for lineage markers specific for B cells and Monocyte/Macrophages, such as CD19- and CD14-cells. Accordingly, CD4+ T cells may be identified as negative for lineage markers specific for CD8 T cells, B cells and Monocyte/Macrophages, such as CD4+, CD8-, CD19-, and CD14-cells.

The selection may be based on semi-quantitative or quantitative detection of the marker. The selection may comprise detecting the presence of the marker to select cells expressing the marker. Alternatively, the selection may comprise detecting the level of expression of the marker to select cells having high levels of the marker. High expression of the marker is determined by comparing the level of expression in the tested cell(s) with a reference. The reference may be a predetermined value or a value obtained with a control cell sample tested in parallel. Typically, the expression level in tested cell(s) is deemed to be higher than the reference if the ratio of the expression level of said marker in said cell(s) to that of the reference is higher than 1.2; for example 1.5, 2, 5; 10, 20 or more. For example, the reference may be a sub-population of CD4+ T cells, for example tested in parallel. The reference may be several different sub-populations of CD4+ T cells, such as the clusters disclosed in the examples herein. The reference may be all the cells contained in the other clusters, except the one analyzed.

The level of expression of proteins in cells is usually defined as “low”, “middle” or “high” using standard criteria that can be applied to any proteins such as the markers disclosed herein. Therefore, a person skilled in the art is able to determine whether a marker as disclosed herein has high expression in a cell.

For example, the precursor cell of the invention may be selected from a population of CD4+ T cells (CD3 and CD4 expressing cells or CD3+, CD4+cells) by selecting the cells expressing FOXP3 (FOXP3+) and/or highly expressing IL2RA (IL2RA^{high}) and further expressing or highly expressing ICOS (ICOS^{high}); preferably expressing ICOS (ICOS+) and highly expressing PD1 (PD1^{high}). T cells may be identified as negative for lineage markers specific for B cells and Monocyte/Macrophages, such as CD19-and CD14-cells. Accordingly, CD4+ T cells may be identified as negative for lineage markers specific for CD8 T cells, B cells and Monocyte/Macrophages, such as CD4+, CD8-, CD19-, and CD14-cells. Therefore, the precursor cell may be defined as having a phenotype comprising: CD3+, CD4+, FOXP3+ and/or IL2RA^{high}, ICOS+and PD1^{high} or CD8-, CD19-, CD14-, CD4+, FOXP3+ and/or IL2RA^{high}, ICOS+and PD1^{high}. The population of CD4+ T cells is preferably isolated from tumor and/or tumor-draining lymph node sample.

Alternatively, the precursor may be produced from induced pluripotent stem cells (iPS) by standard T cell induction techniques that are well-known in the art (Review in Martin U., Front. Med., 2017, doi. org/10.3389).

The precursor cell may be characterized by the expression of further markers. Expression may be high expression or differential expression. High expression is determined by comparing the level of expression of the marker in the tested cell(s) with a reference as disclosed above. Differential expression refers to the specific expression of the marker in the T cell precursor but not in other T cell populations. The expression may be determined at the RNA or protein level and may be semi-quantitative or quantitative. Expression of said markers may be determined by routine techniques in art, such as by single-cell RNA-seq and FACS analysis, for example, as described in the Examples. Antibodies against any of the markers described herein can be used to achieve isolation of the precursor and/or detection of any of the cell markers described herein.

In some embodiments, the T cell precursor further expresses follicular regulatory CD4+ T (TFR) cells markers, Treg markers, and/or tissue-imprinting markers, for example as disclosed in the examples or figures.

In some particular embodiments, the T cell precursor further comprises expression, in particular high expression of at least one of the following markers: CD151, IGFLR1, CD44, SOCS1, CCL20, CLEC2D, CALR, MTHFD2, CD59, MAGEHI, TNFSF13B, LGALS9, THY1, CXCL13, CD38, IL17F, ILIRL1, IL12RB, REL, TCF7, CD200, LRR8D, IL21R, THADA, TOX, TOX2, TNFRSF18, ENTPD1, LAIR2, HDAC9, VDR, IRF4, NR3C1, IGFL2, BATF, ITGA4, ETV6, GEM, MAF, CTLA4, CCR4, TANK, SIRPG, COCH, EBI3, CD28, TGIF1, ICA1, TIGIT, PMCH, SRGN, IL6ST, GEM, SESN1, SEN3, IKZF2, IKZF4, CD82, FYN, CFLAR and ZNF281 genes. The T cell precursor may comprise expression or high expression of one or more or all of the markers listed above.

In some particular embodiments, the T cell precursor further comprises high expression of at least one marker selected from the group consisting of: CTLA-4 (Cytotoxic T-Lymphocyte Associated Protein 4), BATF (Basic Leucine Zipper ATF-Like Transcription Factor), ICA1 (Islet Cell Autoantigen 1), COCH(Cochlin), PMCH (Pro-Melanin Concentrating Hormone), ZNF281 (Zinc Finger Protein 281), THY.1 (Thy-1 Cell Surface Antigen), CD200, IRF4 (Interferon Regulatory Factor 4), TIGIT (T Cell Immunoreceptor With Ig And ITIM Domains), TOX (Thymocyte Selection Associated High Mobility Group Box), PRDM1 (B-Lymphocyte-Induced Maturation Protein 1), CXCL13 (C-X-C Motif Chemokine Ligand 13), BTLA (B-and T-lymphocyte attenuator), CD221(Insulin-like growth factor 1 receptor), TNFRSF18 (Glucocorticoid-induced TNFR-related protein), ITGA4, IL21R (Interleukin 21 Receptor) ), leucine rich repeat containing 32 (LRRC32), Rhotekin 2 (RTKN2), IKAROS Family Zinc Finger 2 (IKZF2), and CD151.

In some particular embodiments, the T cell precursor further comprises high expression of at least one marker selected from the group consisting of: CTLA-4 (Cytotoxic T-Lymphocyte Associated Protein 4), BATF (Basic Leucine Zipper ATF-Like Transcription Factor), ICA1 (Islet Cell Autoantigen 1), COCH(Cochlin), PMCH (Pro-Melanin Concentrating Hormone), ZNF281 (Zinc Finger Protein 281), THY.1 (Thy-1 Cell Surface Antigen), CD200, IRF4 (Interferon Regulatory Factor 4), TIGIT (T Cell Immunoreceptor With Ig And ITIM Domains), TOX (Thymocyte Selection Associated High Mobility Group Box), PRDM1 (B-Lymphocyte-Induced Maturation Protein 1), CXCL13 (C-X-C Motif Chemokine Ligand 13), BTLA (B-and T-lymphocyte attenuator), CD221(Insulin-like growth factor 1 receptor), TNFRSF18 (Glucocorticoid-induced TNFR-related protein), ITGA4, IL21R (Interleukin 21 Receptor) and CD151; in particular selected from the group consisting of: CTLA-4, BATF, ICA1, COCH, PMCH, ZNF281, THY.1, CD200, IRF4 TIGIT, TOX, PRDM1, CXCL13, and BTLA; more particularly CD200 and BTLA. The T cell precursor may comprise high expression of one or more or all of the markers listed above. In some preferred embodiment, the marker is protein.

In some more particular embodiments, the human T cell precursor comprises expression of the markers CD3, CD4 and ICOS; and high expression of the markers IL2RA, CD200, BTLA and PD1. In some other more particular embodiments, the human T cell precursor comprises expression of the markers CD3, CD4, FOXP3 and ICOS; and high expression of the markers CD200, BTLA and PD1. Preferably, the human T cell precursor comprises expression of the markers CD3, CD4 and ICOS; and high expression of the markers IL2RA, CD200, BTLA and PD1. In a particular example, the precursor cell of the invention is selected from a population of CD4+ T cells (CD3 and CD4 expressing cells or CD3+, CD4+ cells) by selecting the cells expressing FOXP3 (FOXP3+) and/or highly expressing IL2RA (IL2RA^{high}); further expressing ICOS (ICOS+); and further highly expressing PD1 (PD1^{high}), CD200 (CD200^{high}) and BTLA (BTLA^{high}). CD4+ T cells may be identified as negative for lineage markers specific for CD8 T cells, B cells and Monocyte/Macrophages, such as CD4+, CD8-, CD19-, and CD14-cells. Therefore, the precursor cell may be defined as having a phenotype comprising: CD3+, CD4+, FOXP3+ and/or IL2RA^{high}, ICOS+, CD200^{high}, BTLA^{high} and PD1^{high} or CD8-, CD19-, CD14-, CD4+, FOXP3+ and/or IL2RA^{high}, ICOS+, CD200^{high}, BTLA^{high} and PD1^{high}.

The T cell precursor can be grown in cell culture medium, in particular supplemented culture medium as known in the art for cell culture. For the expansion of T cell precursor, the culture medium: For example, the X-vivo medium is completed with human serum (10%), b-mercaptoethanol (50 μM), Pen/Strep (1%), non-essential amino acids (1×), and eventually IL-2 (50 IU/mL) and/or with beads coated with anti-human CD3, anti-human CD28, and anti-human CD2.

The T cell precursor differentiates into T cells. In some particular embodiments, the T cell precursor differentiates into both regulatory T cells (Tregs) and conventional T cells (Tconvs).

T cell precursor differentiation into T cells, in particular regulatory T cells (Tregs) and conventional T cells (Tconvs) follows the expression of a specific transcription program comprising upregulation and downregulation of some specific transcription, as disclosed in the examples.

In some particular embodiments, the T cell precursor differentiates into Tregs following expression of a transcription factor program comprising upregulation of at least one of: MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3, HMGA1, YY1, FLI1 and ATF3; and downregulation of at least one of RBPJ, FOXN2, REST, ZNF75A, FOXO3, KLF12, NFATC2, MAF, RUNX2, NR3C1, ZNF281, HIVEP1, HIVEP2, LEF1 and TCF7; preferably comprising upregulation of at least one of: MEF2D, NFE2L2, IRF4, ETV7, REL, HMG20B, IRF5,IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3 and HMGA1; and downregulation of at least one of RBPJ, FOXN2, REST, ZNF75A, FOXO3, KLF12, NFATC2, MAF, RUNX2 and NR3C1; more preferably comprising upregulation of at least one of: IRF1, ID3, NFKB2, NFKB1, HIVEP3 and HMGA1; and downregulation of at least one of NFATC2, MAF, RUNX2 and NR3C1. The transcription program may comprise upregulation and/or downregulation of one or more or all of the transcription factors listed above.

In some particular embodiments, the T cell precursor differentiates into Tconvs following expression of a transcription factor program comprising upregulation of at least one of RORA, PPARG, CREM, NR2C2, ETS1, SP4, CEBPZ, BCL11B, ZNF75D, NFATC2, MAF, RUNX2, NR3C1, YY1, FLI1 and ATF3; and downregulation of at least one of RELB, ZEB1, TOPORS, ZBTB7A, IRF1, ID3, NFKB2, NFKB1, HIVEP3, HMGA1, ZNF281, HIVEP1, HIVEP2, LEF1 and TCF7; preferably comprising upregulation of at least one of RORA, PPARG, CREM, NR2C2, ETS1, SP4, CEBPZ, BCL11B, ZNF75D, NFATC2, MAF, RUNX2 and NR3C1; and downregulation of at least one of RELB, ZEB1, TOPORS, ZBTB7A, IRF1, ID3, NFKB2, NFKB1, HIVEP3 and HMGA1; more preferably comprising upregulation of at least one of NFATC2, MAF, RUNX2 and NR3C1; and downregulation of at least one of IRF1, ID3, NFKB2, NFKB1, HIVEP3 and HMGA1. The transcription program may comprise upregulation and/or downregulation of one or more or all of the transcription factors listed above.

The expression of the transcription factor program may be determined at the RNA or protein level, preferably at the RNA level. Expression of said transcription factors may be determined by routine techniques in art, such as by single-cell RNA-seq, as described in the Examples. Upregulation or downregulation of the transcription factor is determined by comparing the level of expression and the level of accessibility of its binding motif in the tested cell(s) with a reference. The reference may be a predetermined value or a value obtained with a control cell sample tested in parallel. Typically, the expression level in tested cell(s) is deemed to be higher or lower than the reference if the ratio of the expression level of said marker in said cell(s) to that of the reference is higher or lower than 1.2 or more, such as for example 1.5, 2, 5, 10, 20 or more.

Another aspect of the invention relates to a method of differentiation of the precursor cell according to the present disclosure into Tregs, comprising inducing the expression of a transcription factor program comprising upregulation of at least one of MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3, HMGA1, YY1, FLI1 and ATF3; and downregulation of at least one of RBPJ, FOXN2, REST, ZNF75A, FOXO3, KLF12, NFATC2, MAF, RUNX2, NR3C1, ZNF281, HIVEP1, HIVEP2, LEF1 and TCF7 in the cell; preferably comprising upregulation of at least one of: MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3 and HMGA1; and downregulation of at least one of RBPJ, FOXN2, REST, ZNF75A, FOXO3, KLF12, NFATC2, MAF, RUNX2 and NR3C1 in the cell; more preferably comprising upregulation of at least one of: IRF1, ID3, NFKB2, NFKB1, HIVEP3 and HMGA1; and downregulation of at least one of NFATC2, MAF, RUNX2 and NR3C1 in the cell. The method may comprise inducing upregulation and/or downregulation of one or more or all of the transcription factors listed above.

Another aspect of the invention relates to a method of differentiation of the precursor cell according to the present disclosure into Tconvs, comprising inducing the expression of a transcription factor program comprising upregulation of at least one of RORA, PPARG, CREM, NR2C2, ETS1, SP4, CEBPZ, BCL11B, ZNF75D, NFATC2, MAF, RUNX2, NR3C1, YY1, FLI1 and ATF3; and downregulation of at least one of RELB, ZEB1, TOPORS, ZBTB7A, IRF1, ID3, NFKB2, NFKB1, HIVEP3, HMGA1, ZNF281, HIVEP1, HIVEP2, LEF1 and TCF7 in the cell; preferably comprising upregulation of at least one of RORA, PPARG, CREM, NR2C2, ETS1, SP4, CEBPZ, BCL11B, ZNF75D, NFATC2, MAF, RUNX2 and NR3C1; and downregulation of at least one of RELB, ZEB1, TOPORS, ZBTB7A, IRF1, ID3, NFKB2, NFKB1, HIVEP3 and HMGA1 in the cell; more preferably comprising upregulation of at least one of NFATC2, MAF, RUNX2 and NR3C1; and downregulation of at least one of IRF1, ID3, NFKB2, NFKB1, HIVEP3 and HMGA1 in the cell. The transcription program may comprise upregulation and/or downregulation of one or more or all of the transcription factors listed above.

The method of differentiation may be performed in vivo or in vitro. Expression of the transcription factor program may be induced using standard modulators routinely used to modulate gene expression or gene product (protein) activity that are well-known in the art and may be used to modulate transcription factor expression or activity.

In some particular embodiments, the method of differentiation is performed in vitro.

In some particular embodiments, the modulator inhibits or stimulates the activity of the target protein. The modulator of activity may be selected from the group comprising: small organic molecules, aptamers, antibodies, peptides, and other agonists or antagonists such as for example dominant negative mutants or functional fragments of the target protein.

The term “small organic molecule” refers to a molecule of a size comparable to those of organic molecules generally used in pharmaceuticals. The term excludes biological macro molecules (e. g., proteins, nucleic acids, etc.). Preferred small organic molecules range in size up to about 5000 Da, more preferably up to 2000 Da, and most preferably up to about 1000 Da. Various small organic molecule inhibitors or antagonists are known in the art. Identification of new small molecule inhibitors can be achieved according to classical techniques in the field. The current prevailing approach to identify hit compounds is through the use of a high throughput screen (HTS).

Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library, as described in Tuerk C. and Gold L., 1990 and can be optionally chemically modified.

As used herein, the term “antibody” refers to a protein that includes at least one antigen-binding region of immunoglobulin. The antigen binding region may comprise one or two variable domains, such as for example a VH domain and a VL domain or a single VHH or VNAR domain. The term “antibody” encompasses full length immunoglobulins of any isotype, functional fragments thereof comprising at least the antigen-binding region and derivatives thereof. Antigen-binding fragments of antibodies include for example Fv, scFv, Fab, Fab′, F(ab′)2, Fd, Fabc and sdAb (V_{H}H, V-NAR). Antibody derivatives include with no limitation polyspecific or multivalent antibodies, intrabodies and immunoconjugates. Intrabodies are antibodies that bind intracellularly to their antigen after being produced in the same cell (for a review see for example, Marschall AL, Dübel S and Böldicke T “Specific in vivo knockdown of protein function by intrabodies”, MAbs. 2015; 7(6):1010-35). The antibody may be glycosylated. An antibody can be functional for antibody-dependent cytotoxicity and/or complement-mediated cytotoxicity, or may be non-functional for one or both of these activities. Antibodies are prepared by standard methods that are well-known in the art such as hybridoma technology, selected lymphocyte antibody method (SLAM), transgenic animals, recombinant antibody libraries or synthetic production.

In some other particular embodiments, the modulator inhibits the expression of the target. In some preferred embodiments, the inhibitor is selected from the group comprising: anti-sense oligonucleotides, interfering RNA molecules, ribozymes and genome or epigenome editing enzyme complexes.

Anti-sense oligonucleotides are RNA, DNA or mixed and may be modified. Interfering RNA molecules include with no limitations siRNA, shRNA and miRNA. Genome and Epigenome editing complex may be based on any known system such as CRISPR/Cas, TALENs, Zinc-Finger nucleases and meganucleases. Anti-sense oligonucleotides, interfering RNA molecules, ribozymes, genome and epigenome editing complexes are well-known in the art and inhibitors of the target according to the invention may be easily designed based on these technologies using the sequences of the targets that are well-known in the art.

The above-disclose modulators may be used to modulate expression or activity of the various transcription factors and markers (cell markers) disclosed herein.

In some embodiments of the methods according to the invention, the modulation of transcription factors involved in the differentiation may alter the stability, phenotype, and or tissue retention properties of blood derived Tregs and Tconvs.

The differentiated regulatory T cells (Tregs) and/or conventional T cells (Tconvs) can be grown in cell culture medium, in particular supplemented culture medium as known in the art for cell culture. In some embodiments, the differentiated regulatory T cells (Tregs) and/or conventional T cells (Tconvs) are further expanded in vitro. The culture medium as described above for the T cell precursor may be adapted for the expansion of Treg or Tconv cells, for example increasing IL-2 (3000 IU/mL) concentration.

In some particular embodiments, the regulatory T cells (Tregs) and/or conventional T cells (Tconvs) are tumor-specific, tumor-infiltrating and/or migrate between lymph nodes and tumor. In some preferred embodiments, the regulatory T cells (Tregs) and/or conventional T cells (Tconvs) express tissue-imprinting markers or are tissue imprinted cells as disclosed herein.

The inventors have also identified molecular signatures of migrating and resident Tregs and Tconvs in the tumors and lymph nodes (LN) that can be used to modulate migration and homing of Tregs and Tconvs in the tumor.

Therefore, another aspect of the invention relates to a molecular signature of migrating or resident regulatory T cells or conventional T cells in the tumor or lymph node selected from the group consisting of:
A signature of migrating regulatory T cells present in the lymph node comprising the expression of CCR8, HAVCR2, IL2RB, and LAIR2 genes;
A signature of migrating conventional T cells present in the the lymph node comprising the expression of GLUL, HLA-A, HLA-DRA, NKG7 and SEC61G genes;
A signature of migrating regulatory T cells present in the Tumor comprising the expression of: CCR8, CTLA4 and SDC4 genes;
A signature of migrating conventional T cells present in the tumor comprising the expression of: CLEC2B, and HLA-DQA1 genes;
A signature of resident regulatory T cells present in the lymph node comprising the expression of: CCR7 and LEF1 genes;.
A signature of resident conventional T cells present in the lymph node are comprising the expression of: CCR7, LEF1, SELL, SESN1 and TCF7 genes;
A signature of resident regulatory T cells present in the Tumor comprising the expression of: ICAM3, IL12RB2, IL27RA, LAMP1, LGALS3, TNFRSF14, TNFRSF18 and TNFRSF4 genes;
A signature of resident conventional T cells present in the tumor comprising the expression of: aCALM1, CALM3, ICAM3, IL6ST, LGALS3, SIRPG, BTLA and CXCL13 genes.

Another aspect of the invention relates to a screening method for inducers of differentiation of a T cell precursor according to the present disclosure into Tregs or Tconvs, comprising: (a) administering a modulator as disclosed herein to a T cell precursor and (b) measuring the level of expression of at least one transcription factor of the transcription factor program for Treg or Tconv differentiation as listed above, in the T cell precursor, and (c) identifying a modulator that upregulates a transcription factor that is upregulated in the transcription factor program or downregulates a transcription factor that is downregulated in the transcription factor program, in the treated precursor cell as compared to untreated precursor cell (control).

The invention encompasses the modified cells derived from the T cell precursor and differentiated Treg and Tconv therefrom. In some particular embodiments the modified T cell, in particular Treg or Tconv, is genetically engineered, notably to express an engineered receptor such as a chimeric antigen receptor (CAR-T cells) or exogenous or modified TCR (TCR-T cells) specific for a target antigen. Such a genetically engineered receptor can be used to graft the specificity of a monoclonal antibody or specific TCR for a given antigen onto effector T cells.

The invention also encompasses the use of the TCR from imprinted Tregs and Tconvs as disclosed herein to generate TCR T-cells, as well as the TCR-T cells comprising an engineered TCR from imprinted Tregs and Tconvs as disclosed herein.

The invention also relates to a CAR-T cell or a TCR-T cell modified to express tissue-imprinting markers as described herein. In particular, the CAR-T cells or TCR-T cell may be modified to stimulate expression of markers of tissue residency such as CXCR3, CXCR6, CD69, IL-1RL1, PRDM1 and others as disclosed in the examples or figures of the present disclosure; in particular expressing markers of tissue residency such as C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1 (ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT, and/or (ii) inhibit expression of markers of blood circulation, such as CCR7, SELL, S1PR1, and KLF2, and others as disclosed in the examples or figures of the present disclosure. The inhibition or stimulation of expression of the markers may be obtained by contacting the CAR-T cell or TCR-T cell with a modulator as disclosed herein. Alternatively, the CAR-T cell or TCR-T cell may be genetically engineered to insert at least one transgene expressing a marker of residency (knock-in) and/or inactivate at least one gene encoding a marker of blood circulation (knock-out) using standard genome engineering techniques that are well-known in the art, preferably using gene editing systems such as CRISPR/Cas, TALEN and meganucleases.

The invention further relates to the molecular signatures of migration and tissue-residency of Tconvs or Tregs, in particular in tumors and lymph nodes that are disclosed herein. The invention encompasses the use of modulators of said molecular signatures to modulate immune infiltration of a diseased tissue, in particular a tumor by favoring the tissue homing and/or migration in the tumor of effector Tconvs or impairing the tissue homing and/or migration in the tumor of Tregs.

### Therapeutic Applications

The T cell precursor, the Treg or Tconv differentiated from the T cell precursor, and the inducers of differentiation of said T cell precursor, Treg or Tconv according to the present disclosure are useful in immunotherapy of various diseases such as cancer, infectious or immune diseases.

The inducer of differentiation of said regulatory of conventional T cells may be obtained using the screening method according to the present disclosure.

The therapeutic uses according to the present disclosure comprise adoptive T cell therapy which may be combined with another therapy. Adoptive cell therapy (ACT) also called adoptive T cell therapy, adoptive cell transfer, cellular adoptive immunotherapy or T-cell transfer therapy is a type of immunotherapy in which immune cells, in particular T cells, are administered to a patient to help the immune system fight diseases such as cancer, infectious diseases and others. The immune cells used in adoptive cell therapy may be autologous or allogenic immune cells. Allogenic refers to histocompatible (HLA-compatible) cells. Immune cells for Adoptive cell therapy (ACT) may be engineered to recognize an antigen of interest for therapy (redirected T cell immunotherapy, CAR T cell therapy). The immune cells for adoptive T cell therapy may be delivered to the individual in need thereof by any appropriate mean such as for example by intravenous injection (infusion or perfusion), or injection in the tissue of interest (implantation).

The T cell precursor, Treg, Tconv and/or inducer of differentiation according to the present disclosure may be used for the treatment of various diseases, in particular, cancer, acute or chronic inflammatory diseases, autoinflammatory diseases, miscarriage, allergic diseases, autoimmune or infectious diseases, graft-versus-host disease and graft-rejection, and tissue repair (wound healing).

### Tconv Are Used in Particular for the Treatment of Cancer and Infectious Diseases.

Treg used in particular for the treatment acute or chronic inflammatory diseases, autoinflammatory diseases, miscarriage, allergic diseases, autoimmune, graft-versus-host disease and graft-rejection and tissue repair (wound healing).

Non-limiting examples of autoimmune diseases include: type 1 diabetes, rheumatoid arthritis, psoriasis and psoriatic arthritis, multiple sclerosis, Systemic lupus erythematosus (lupus), Inflammatory bowel disease such as Crohn's disease and ulcerative colitis, Addison's disease, Grave's disease, Sjögren's disease, alopecia areata, autoimmune thyroid disease such as Hashimoto's thyroiditis, myasthenia gravis, vasculitis including HCV-related vasculitis and systemic vasculitis, uveitis, myositis, pernicious anemia, celiac disease, Guillain-Barre Syndrome, chronic inflammatory demyelinating polyneuropathy, scleroderma, hemolytic anemia, glomerulonephritis, autoimmune encephalitis, fibromyalgia, aplastic anemia and others. Non-limiting examples of inflammatory and allergic diseases include: neuro-degenerative disorders such as Parkinson disease, chronic infections such as parasitic infection or disease like Trypanosoma cruzi infection, allergy such as asthma, atherosclerosis, chronic nephropathy, and others. The disease may be allograft rejection including transplant-rejection, graft-versus-host disease (GVHD) and spontaneous abortion.

Infectious diseases include viral, bacterial, fungal and parasitic diseases.

As used herein, the term “cancer” refers to any cancer that may affect any one of the following tissues or organs: breast; liver; kidney; heart, mediastinum, pleura; floor of mouth; lip; salivary glands; tongue; gums; oral cavity; palate; tonsil; larynx; trachea; bronchus, lung; pharynx, hypopharynx, oropharynx, nasopharynx; esophagus; digestive organs such as stomach, intrahepatic bile ducts, biliary tract, pancreas, small intestine, colon; rectum; urinary organs such as bladder, gallbladder, ureter; rectosigmoid junction; anus, anal canal; skin; bone; joints, articular cartilage of limbs; eye and adnexa; brain; peripheral nerves, autonomic nervous system; spinal cord, cranial nerves, meninges; and various parts of the central nervous system; connective, subcutaneous and other soft tissues; retroperitoneum, peritoneum; adrenal gland; thyroid gland; endocrine glands and related structures; female genital organs such as ovary, uterus, cervix uteri; corpus uteri, vagina, vulva; male genital organs such as penis, testis and prostate gland; hematopoietic and reticuloendothelial systems; blood; lymph nodes; thymus.

The term “cancer” according to the invention comprises leukemias, seminomas, melanomas, teratomas, lymphomas, non-Hodgkin lymphoma, neuroblastomas, gliomas, adenocarcinoma, mesothelioma (including pleural mesothelioma, peritoneal mesothelioma, pericardial mesothelioma and end stage mesothelioma), rectal cancer, endometrial cancer, thyroid cancer (including papillary thyroid carcinoma, follicular thyroid carcinoma, medullary thyroid carcinoma, undifferentiated thyroid cancer, multiple endocrine neoplasia type 2A, multiple endocrine neoplasia type 2B, familial medullary thyroid cancer, pheochromocytoma and paraganglioma), skin cancer (including malignant melanoma, basal cell carcinoma, squamous cell carcinoma, Kaposi's sarcoma, keratoacanthoma, moles, dysplastic nevi, lipoma, angioma and dermatofibroma), nervous system cancer, brain cancer (including astrocytoma, medulloblastoma, glioma, lower grade glioma, ependymoma, germinoma (pinealoma), glioblastoma multiform, oligodendroglioma, schwannoma, retinoblastoma, congenital tumors, spinal cord neurofibroma, glioma or sarcoma), skull cancer (including osteoma, hemangioma, granuloma, xanthoma or osteitis deformans), meninges cancer (including meningioma, meningiosarcoma or gliomatosis), head and neck cancer (including head and neck squamous cell carcinoma and oral cancer (such as, e.g., buccal cavity cancer, lip cancer, tongue cancer, mouth cancer or pharynx cancer)), lymph node cancer, gastrointestinal cancer, liver cancer (including hepatoma, hepatocellular carcinoma, cholangiocarcinoma, hepatoblastoma, angiosarcoma, hepatocellular adenoma and hemangioma), colon cancer, stomach or gastric cancer, esophageal cancer (including squamous cell carcinoma, larynx, adenocarcinoma, leiomyosarcoma or lymphoma), colorectal cancer, intestinal cancer, small bowel or small intestines cancer (such as, e.g., adenocarcinoma lymphoma, carcinoid tumors, Kaposi's sarcoma, leiomyoma, hemangioma, lipoma, neurofibroma or fibroma), large bowel or large intestines cancer (such as, e.g., adenocarcinoma, tubular adenoma, villous adenoma, hamartoma or leiomyoma), pancreatic cancer (including ductal adenocarcinoma, insulinoma, glucagonoma, gastrinoma, carcinoid tumors or vipoma), ear, nose and throat (ENT) cancer, breast cancer (including HER2-enriched breast cancer, luminal A breast cancer, luminal B breast cancer and triple negative breast cancer), cancer of the uterus (including endometrial cancer such as endometrial carcinomas, endometrial stromal sarcomas and malignant mixed Müllerian tumors, uterine sarcomas, leiomyosarcomas and gestational trophoblastic disease), ovarian cancer (including dysgerminoma, granulosa-theca cell tumors and Sertoli-Leydig cell tumors), cervical cancer, vaginal cancer (including squamous-cell vaginal carcinoma, vaginal adenocarcinoma, clear cell vaginal adenocarcinoma, vaginal germ cell tumors, vaginal sarcoma botryoides and vaginal melanoma), vulvar cancer (including squamous cell vulvar carcinoma, verrucous vulvar carcinoma, vulvar melanoma, basal cell vulvar carcinoma, Bartholin gland carcinoma, vulvar adenocarcinoma and erythroplasia of Queyrat), genitourinary tract cancer, kidney cancer (including clear renal cell carcinoma, chromophobe renal cell carcinoma, papillary renal cell carcinoma, adenocarcinoma, Wilms tumor, nephroblastoma, lymphoma or leukemia), adrenal cancer, bladder cancer, urethra cancer (such as, e.g., squamous cell carcinoma, transitional cell carcinoma or adenocarcinoma), prostate cancer (such as, e.g., adenocarcinoma or sarcoma) and testis cancer (such as, e.g., seminoma, teratoma, embryonal carcinoma, teratocarcinoma, choriocarcinoma, sarcoma, interstitial cell carcinoma, fibroma, fibroadenoma, adenomatoid tumors or lipoma), lung cancer (including small cell lung carcinoma (SCLC), non-small cell lung carcinoma (NSCLC) including squamous cell lung carcinoma, lung adenocarcinoma (LUAD), and large cell lung carcinoma, bronchogenic carcinoma, alveolar carcinoma, bronchiolar carcinoma, bronchial adenoma, lung sarcoma, chondromatous hamartoma and pleural mesothelioma), sarcomas (including Askin's tumor, sarcoma botryoides, chondrosarcoma, Ewing's sarcoma, malignant hemangioendothelioma, malignant schwannoma, osteosarcoma and soft tissue sarcomas), soft tissue sarcomas (including alveolar soft part sarcoma, angiosarcoma, cystosarcoma phyllodes, dermatofibrosarcoma protuberans, desmoid tumor, desmoplastic small round cell tumor, epithelioid sarcoma, extraskeletal chondrosarcoma, extraskeletal osteosarcoma, fibrosarcoma, gastrointestinal stromal tumor (GIST), hemangiopericytoma, hemangiosarcoma, Kaposi's sarcoma, leiomyosarcoma, liposarcoma, lymphangiosarcoma, lymphosarcoma, malignant peripheral nerve sheath tumor (MPNST), neurofibrosarcoma, plexiform fibrohistiocytic tumor, rhabdomyosarcoma, synovial sarcoma and undifferentiated pleomorphic sarcoma, cardiac cancer (including sarcoma such as, e.g., angiosarcoma, fibrosarcoma, rhabdomyosarcoma or liposarcoma, myxoma, rhabdomyoma, fibroma, lipoma and teratoma), bone cancer (including osteogenic sarcoma, osteosarcoma, fibrosarcoma, malignant fibrous histiocytoma, chondrosarcoma, Ewing's sarcoma, malignant lymphoma and reticulum cell sarcoma, multiple myeloma, malignant giant cell tumor chordoma, osteochronfroma, osteocartilaginous exostoses, benign chondroma, chondroblastoma, chondromyxoid fibroma, osteoid osteoma and giant cell tumors), hematologic and lymphoid cancer, blood cancer (including acute myeloid leukemia, chronic myeloid leukemia, acute lymphoblastic leukemia, chronic lymphocytic leukemia, myeloproliferative diseases, multiple myeloma and myelodysplasia syndrome), Hodgkin's disease, non-Hodgkin's lymphoma and hairy cell and lymphoid disorders, and the metastases thereof.

In some embodiments the cancer is selected from the group comprising: non-small cell lung cancer (NSCLC); breast, skin, ovarian, kidney and head and neck cancers; and rhabdoid tumors; preferably non-small cell lung cancer (NSCLC) and breast cancer.

In particular, the invention relates to a pharmaceutical composition comprising a T cell precursor according to the present disclosure, a Treg or a Tconv differentiated from the T cell precursor, and/or an inducer of differentiation of the T cell precursor, Treg or Tconv as an active component.

In the various embodiments of the present invention, the pharmaceutical composition comprises a therapeutically effective amount of the T cell precursor, Treg, Tconv and/or inducer of differentiation. In the context of the invention a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect. The effective dose is determined and adjusted depending on factors such as the composition used, the route of administration, the physical characteristics of the individual under consideration such as sex, age and weight, concurrent medication, and other factors, that those skilled in the medical arts will recognize. The effective dose can be determined by standard clinical techniques. In addition, in vivo and/or in vitro assays may optionally be employed to help predict optimal dosage ranges.

In the various embodiments of the present invention, the pharmaceutical composition comprises a pharmaceutically acceptable carrier and/or vehicle. The pharmaceutical vehicles and carriers are those appropriate to the planned route of administration, which are well known in the art. The pharmaceutical composition is formulated for administration by a number of routes, including but not limited to parenteral and local. The pharmaceutical composition of the present invention is generally administered according to known procedures, at dosages and for periods of time effective to induce a therapeutic effect in the patient. The pharmaceutical composition may be administered by any convenient route, such as in a non-limiting manner by injection, perfusion or implantation. The administration can be systemic, local or systemic combined with local. Systemic administration is preferably intravascular such as intravenous (IV) or intraarterial; intraperitoneal (IP) or else. In some preferred embodiments, the administration is parenteral, preferably intravascular such as intravenous (IV) or intraarterial. The parenteral administration is advantageously by injection or perfusion.

The pharmaceutical composition may also comprise an additional therapeutic agent, in particular an agent useful for the treatment of a disease according to the present disclosure.

The additional therapeutic agent is preferably an antigen specific of the disease or an anticancer, anti-infectious or immunomodulatory agent.

The T cell precursor, Treg, Tconv and/or inducer of differentiation, or pharmaceutical composition of the invention may be used in combination with another therapy, wherein the combined therapies may be simultaneous, separate or sequential.

The additional therapy is in particular an anticancer, anti-infectious therapy and/or immunotherapy. Anti-infectious therapy includes the use of the known antibacterial, antiviral, antiparasitic, antifungal agents currently used for treating infectious diseases. Anticancer therapy includes chemotherapy, targeted therapy, radiotherapy, anticancer vaccine and immunotherapy including immune checkpoint inhibitors, co-stimulatory antibodies, and CAR-T cell therapy.

Another aspect of the invention relates to the T cell precursor, Treg, Tconv, inducer of differentiation, or pharmaceutical composition according to the present disclosure as a medicament, in particular for use in the treatment of a disease according to the present disclosure.

The invention provides also a method for treatment of a patient in need thereof, comprising:
Providing autologous or allogenic T cell precursor, or Treg or Tconv differentiated from the T cell precursor according to the present disclosure; and
Administering the T cell precursor, or Treg or Tconv differentiated from the T cell precursor to the patient.

In particular embodiments, the T cell is specific for an antigen of interest, preferably a tumoral antigen or microbial antigen such as viral antigen. In particular embodiments, the T cell is expanded, modified and/or engineered before administration to the subject.

The various embodiments of the present disclosure can be combined with each other and the present disclosure encompasses the various combinations of embodiments of the present disclosure.

The practice of the present invention will employ, unless otherwise indicated, conventional techniques which are within the skill of the art. Such techniques are explained fully in the literature.

The invention will now be exemplified with the following examples, which are not limitative, with reference to the attached drawings in which:

### BRIEF DESCRIPTION OF THE DRAWINGS

FIG. 1. scRNA-seq and scATACseq landscape of paired blood, TDLNs and tumor samples from NSCLC patients reveal a common gene regulatory network imprinting tissue residency. A) UMAP projection of cells obtained from scRNA-seq integrated data (3 tissues, 5 patients: 15 samples). Dots represent individual cells colored by cluster identity, and are classified in 3 groups: Tregs, Tconvs, and Treg/Tconv mix (Tmix) clusters. B) UMAP plots displaying expression levels of FOXP3, IL2RA, CD40LG, IL7R genes (gray: low expression level; red: high expression level). C) Density gradients representing per-tissue distribution of cells in the UMAP. D) UMAP projection of integrated scATAC-seq nuclei from 6 samples (3 tissues, 2 patients). Dots represent individual nuclei colored by cluster identity, and are separated in 3 groups: Tregs, Tconvs, and TFH-like cells. E) UMAP plots displaying the gene score levels (Blue=low expression level; Yellow=high expression level) of 6 pathognomonic genes characterizing Tregs, Tconvs and Follicular-like cells. F) Density gradients representing per-tissue distribution of cells in the UMAP. G) Volcano plot of maximum motif delta (defined as TF motif deviation score driving variation among clusters, y-axis), and the correlation value between the gene expression level (calculated from the integrated scRNA-seq and scATAC-seq data) and the TF motif enrichment (ME, x-axis). Colored are the 25% most variable TFs; positive regulators in red and negative regulators in blue. H) UMAPs plots displaying gene expression (from scRNA-seq and scATAC-seq integration), and ChromVAR TF deviation score (TF motif enrichment) of BATF. I) FACS analysis: Geometric means of BATF protein expression across clusters (numbers in black) and corresponding fluorescence-minus-one controls (FMO) (numbers in grey), from a representative LN samples of free-of treatment NSCLC patient. J) Heatmap displaying TF motif enrichment (filtered by FDR<0.05, ME>1) of inferred positive regulators selected from A, across clusters. Columns and rows are ordered according to hierarchical clustering.

FIG. 2 . Tcr Sequencing Reveals Tumor Clonal Expansions and Lns-tumors Migration f tissue imprinted cells. A) Heatmap representing normalized Z-score of Gini-Index from each cluster/tissue. The tumor expanded clusters are highlighted. B) Projection onto UMAPs from each tissue of cells from tumor-expanded clones C), or from clones bearing a NeoTCR-D) Scatter plots visualizing Treg/Tconv shared TCR alpha/beta clones. Dot size is proportional to the Normalized Morisita-Horn Index (MHI) of intersected groups, calculated either among total Tregs (green triangle), or Tconvs (red triangle) in blood, lymph node or tumor. E) Top: Scheme illustrating the definition of resident and migrating clones. Bottom panels: Log₂FC/FC plots comparing migrating and resident cells per tissue (Log2FC>0.2 and adj-pVal<0.05). Selected genes from each comparison are displayed in the corresponding squares.

FIG. 3. Multiomic CD4+ T cell profiling uncover the progenitor potential of Treg follicular-like cells. A) Projection of all tissue cells and RNA velocity vectors (streamlines) from all clusters (left) in a UMAP graph or selected clusters in a diffusion map (right). B) Volcano plot showing selected differentially expressed genes from integrated data (scATAC-seq+scRNA-seq), between Treg-FL and all other clusters (Log2FC>0.2, and adj.pVal<0.05). C) FACS analysis performed on LN and tumor samples from a representative free-of treatment NSCLC patient. Dot plots showing the gating strategy used to define the five tissue imprinted and naïve subpopulations; numbers indicate percentage of cells in the corresponding gate. D) FACS analysis: of protein expression of selected features across subsets and corresponding fluorescence-minus-one controls (FMO) (numbers in grey), from a representative LN sample of free-of treatment NSCLC patient. Geometric means are indicated (numbers in black).

FIG. 4. TF: gene regulatory programs explain the alternative developmental pathways of Treg-FL. A) A-B) UMAP visualization of pseudo-time defined vector ordering the nuclei across the selected trajectory: (A) From Treg-FL to Treg-E, and (B) from Treg-FL to Tconv-KLRB1. Panels below UMAP plots display the gene integrated expression of FOXP3 across pseudo-time. Heatmaps (right) display the correlated GI and TF's motif enrichment of the TF explaining the trajectory across the pseudo-time. C) Venn diagram of TFs being lost (yellow and blue) or gained (green and red) across the trajectories in A-B.

FIG. 5. Gate strategy for Treg-FL precursor live isolation. A) Gating strategy to isolate precursor Treg-FL as CD200high, BTLAhigh, ICOS+, PD1high from live CD4+CD8-CD14-CD19-CD25high cells from lymph nodes and tumors of two NSCLC patients (representative data of 7 patients). B) Proportion of precursor Treg-FL in LNs and Tumors from two NSCLC patients.

FIG. 6. IRF1 is more highly expressed in blood and tumor-resident Tregs compared to Tconv cells, and its deletion results in a lower migration and inhibitory potential. A) IRF1 protein expression in human Tconvs and Tregs from healthy donor PBMCs non stimulated or stimulated in vitro with 1000 IU/mL of IFNγ for 18 hours, and tumor from breast cancer patients. MFI=Mean Fluorescence Intensity. B) Western blot quantification of IRF1 expression in FACS sorted Tregs and Tconvs from healthy donor peripheral blood after expansion and electroporation with CRISPR-Cas9 RNP targeting IRF1. C) Schematic representation of the experiment with humanized mice. To induce graft-versus-tumor disease in humanized mouse model, MDA-MB231 human tumor cells were engrafted subcutaneously in the flank of immunodeficient Nod Scid Gamma (NSG) mice. 10 days after (when tumors were palpable), PBMCs were injected intravenously together with WT (black) or IRF1 KO (violet) Tconvs (green) or Tregs (red). To distinguish by flow cytometry PBMCs from Tconvs or Tregs, donors were selected to be HLA-A2+ for PBMCs and HLA-A2-for Tconvs or Tregs. D) Tumor weight at day 16 for the different groups. E) Percentage of WT (black) and IRF1 KO (violet) Tconvs and Tregs (gated on HLA-A2-) among infiltrated human CD45+ cells in the tumor. F) Representative dot plots of Treg-marker expression in WT and IRF1 KO Tregs from the tumor of NSG mice. Statistical analyses were performed using a paired t-test corrected for multiple comparisons. *: p<0.05; **: p<0.01; ***: p<0.001

### EXAMPLES

### Material and Methods

### Clinical Sample Collection

Matched samples of blood, tumor-draining LNs and tumors were collected from patients with NSCLC and luminal breast cancer having undergone standard-of-care surgical resection at the Institut Mutualiste Mountsouris (Paris, Francia). Tissue samples were taking from surgical residues available after histopathological analysis and not required for diagnosis. The human experimental procedures follow the Declaration of Helsinki guidelines and were approved by the Institutional Review Board and Ethics Committee of Institut Curie Hospital group (CRI-DATA190154). All patients signed a written consent. Samples were characterized by IHC, NGS and detection of genomic abnormalities by Cytoscan. Blood buffy coats from healthy donors were collected at the Etablissement Français du Sang.

### Samples and Cell Isolation

Samples were processed within 4 hours after the primary surgery, cut into small fragments, and digested with 0.1 mg/ml Liberase TL (Roche) in the presence of 0.1 mg/ml DNase (Roche) for 30 min before the addition of CO2 independ medium (GIBCO). Cells were then filtered and mechanically dissociated with a 2.5 ml syringe's plunger on a 40-μm cell strainer (BD) and wash with CO2 independent medium (GIBCO) 0.4% human BSA. Peripheral blood mononuclear cells (PBMC) were obtained by ficoll gradient centrifugation of the buffy coats using ficoll tubes (Stemcell).

### FACs-Sorting and ScRNA-seq and scATAC-seq

For each tissue, Tregs (DAPI-CD45+ CD4+ CD25hi CD127lo) and Tconvs (DAPI-CD45+ CD4+ CD25lo CD127lo/hi) were FACS-sorted and admixed at a fifty/fifty ratio before loading on a 10× Chromium (10× Genomics). For the first 2 patients, libraries were prepared using a Single Cell 3′ Reagent Kit (V2 chemistry, 10× Genomics); for the following 3 patients, libraries were prepared using the Single Cell 5′ Reagent kit (Immunoprofiling Kit, 10× Genomics), with an additional step to enrich for V(D)J reads according to the manufacturer's protocol; and for the 6 scATAC seq samples (patients 6 and 7), nuclei were isolated and transposition and further steps were performed following manufacturer's instructions (Chromium Single Cell ATAC Reagent Kits). In the three protocols, the chip was loaded to recover 10,000 cells/nuclei (5,000 Tregs and 5,000 Tconvs) per sample. Indexed libraries were tested for quality, equimolarly pooled and sequenced with Illumina NovaSeq using paired-end 26×98 bp as sequencing mode (Transcriptome or Gene Expression), targeting at least 50,000 reads per cell. The single cell TCR amplification and sequencing was performed after 5′GEX generation using the Single Cell V(D)J kit according to the manufacturer's instructions (10× Genomics). Briefly, V(D)J segments were enriched from amplified cDNA by two human TCR target PCRs, followed by the specific library construction. The TCR enriched cDNA and the library quantification and quality assessment were achieved using a dsDNA High Sensitivity Assay Kit and Bioanalyzer Agilent 2100 System. V(D)J libraries were sequenced on an Illumina Hiseq or Miseq using paired-end 150 bp as sequencing mode. Finally, indexed ATAC-seq libraries were tested for quality, equimolarly pooled and sequenced with Illumina NovaSeq, targeting at least 50,000 reads per nucleus.

### Isolation of Tregs and Tconvs From Periphereal Blood

For Tconv purification, CD4+ cells were enriched from total PBMCs by negative selection with the CD4 T cell isolation kit (Miltenyi). For Treg purification, CD25+ cells were isolated from total PBMCs by positive selection with the CD25 Microbeads II kit (Miltenyi). Recovered CD4+ and CD25+ cells were stained with Live-Dead fixable dye (Aqua), anti-CD4, anti-CD8, anti-CD25 and anti-CD127 (Table 1). Cells were resuspended at 10.106cell/mL in PBS with EDTA (2mM) and Bovine Fetal Serum (BSA, 0.5%). Tconvs were FACS sorted from CD4+ cells as Aqua-CD4+CD127+CD25low while Tregs were FACS sorted from CD25+ cells as Aqua-CD4+CD127-CD25high using BD FACSAria™ III Sorter. The sorted cells were collected in X-vivo media (Ozyme), centrifugated and resuspended at 106 cell/mL in X-vivo media completed with IL-2 (300IU/mL, Miltenyi) and with soluble αCD3αCD28αCD2 (25 μL/mL, Stemcell) for genome edition or with beads αCD3αCD28(1bead: 1cell, Thermofisher) for expansion. The X-vivo media was completed with human serum (10%, Corning), β-mercaptoethanol (50 μM, Gibco), Penicillin/Streptavidin (1%, Fisher), non-essential amino acids (1×, Gibco). To expand sorted Tregs and Tconvs, 300 IU/mL of fresh human IL-2 was added every 3 days and reactivation was performed every week.

### Pre-Processing of ScRNAseq

Raw base call (BCL) files produced by Illumina sequencer were demultiplexed and converted into Fastq files using cellranger mkfastq function from Cellranger version 2.1.1 with default parameters and bcl 2 fastq2 version 2.20. Generated Fastq files were processed using Cellranger version 3.0.2, that introduces an important improvement based on the EmptyDrops method to identify population of low RNA content cells. Cellranger count was run on each Fastq file based on 10×Genomics provided hg 38/GRCh38 human reference genome (refdata-cellranger-GRCh 38-1.2.0 ).

### ScRNA-seq Analysis

Filtering of data: downstream analysis was done using R package Seurat version 3.1.1-3.2.3 with R version 3.6.1-3.6.2⁴⁴. After imported output files produced by cellranger into seurat pipeline, some filters were applied:
Cells with fewer than 200 genes were removed to filters debris, death cells and other cells with few genes.
A second filter has applied to remove others uninformative cells and possible doublets cells using a process based on percentage of mitochondrial genes and total counts of UMI by cell. In details, 1) the polymodal distribution of the cell counts was characterized by log2 percentage of mitochondrial genes and the polymodal distribution of cell counts by log2 total count of UMI by cell; 2) for each of these distributions, the two maxima values of the polymodal distribution was algebraically determined to identify the lowest minimum value between them. These minimum values will be use as cutoff to determine the uninformative cells. In the cases which no minimum could be identified for the distribution for mitochondrial genes, 10% was used as cutoff; 3) cells were filtered with percentages of mitochondrial genes higher than the cutoff defined by the polymodal distribution and with a total count of UMI by cell lower than the cutoff defined by the polymodal distribution of UMI by cell.

Elimination of Contaminant cells: data were normalized sample by sample using global-scaling normalization (using NormalizeData function with “LogNormalize” as normalization method) and the 2,000 most highly variable genes were identified for each sample (using FindVariableFeatures with “vst” as method, with low-and high-cutoffs for feature dispersions fixed at 0.5 and Inf and with low-and high-cutoffs for feature means fixed at-Inf and Inf). Integration process to merge the 3 different tissues of the 5 different patients was performed with the identified anchors (using FindIntegrationAnchors followed by IntegrateData, both using the top 30 PCs). Integrated data was scaled (using ScaleData). To reduce the technical noise, principal component analysis (PCA) was performed to work on the most contributing principal components (PCs). Graph-based clusterization was done at different resolutions (using FindNeighbors on the first fifty PCs and FindClusters for the resolution between 0 and 2 for each decimal) and visualized using Clustree version 0.2.2⁴⁵. UMAP reduction was performed (using RunUMAP on the first fifty PCs) to visualize the data in UMAP projection. Clustering with resolution 0.1 was satisfying for the identification of contaminant cell based on absence of expression of T cell markers (CD3E, CD3G, TRAC, TRBCA and TRBC2) and expression of other immune population markers (CD79A for B cells, CD14 for monocytes, CD11C for dendritic cells).

Integration: once identified contaminant cells were removed from data obtained after filters (Filtering of data step), and the analysis described above was repeated: normalization for each sample followed by highly variable gene identification. Then, samples were merged using integration process, data was scaled, graph-based clusterization was done and UMAP reduction was performed (as described above).

Selection of resolution: clusterization obtained at resolution 1.1 was used for downstream analysis after merging clusters C2 with C19 (Tconv_N).

Differential analysis: to characterize identities of cell clusters, differentially expressed genes among clusters were identified with MAST⁴⁶ (using FindAllMarkers and returning only positive markers with a minimum log Fold-Change (logFC) of 0.2 and a minimum fraction of cells expressing the gene in either of the two groups (min.pct) fixed at 0.05).

Signatures: in addition of differential analysis, signatures from public data were used (using AddModuleScore using half of the features composing the observed signature as control).

Reassignment: for “T-mix” clusters, which displayed a mixture of Treg and Tconv profiles, the transfer label function was used to differentiate cells between Tconv-like and Treg-like (using TransferData with the 30 most contributing PCs).

IPA: pathway analyses of differential expressed genes were uploaded on Ingenuity Pathway Analysis (Ingenuity® Systems, www. ingenuity. com) for the analysis of “Disease and bio-function”, “Canonical pathway”, “Causal network”, and “Up-stream regulators”. Pathways were considered significantly when the overlap p≤0.05.

Slingshot: to reconstruct a developmental path among blood, LN and tumor cells, trajectories were inferred using Slingshot version 2.2.1 with R version 4.1.3, using the 2,000 integrated genes.

Velocity analysis: to investigate developmental dynamics in the data, Velocyto version 0.17.17⁴⁷ with python version 3.6.2 was used to annotate reads between spliced, unspliced and ambiguous genes for each sample individually. Obtained loom files were processed using SeuratWrappers version 0.3.0, Seurat version 4.0.4, SeuratDisk version 0.0.0.9019 and velocyto. R version 0.6, with R version 4.1.1 and combined by tissue. For downstream analysis, RNA velocities were computed on python version 3.9.5 with scVelo dynamical model (version 0.2.3⁴⁸ using the 2,000 most variable genes from h5ad files generated from LNs and tumors separately.

### ScTCRseq Analysis

Pre-processing of scTCRseq: Fastq files were produced from raw base call (BCL) files using cellranger mkfastq function from Cellranger version 2.1.1 with default parameters and bcl2 fastq2 version 2.20. To produce TRA and TRB V(D)J sequence assembly, each generated Fastq files was processed using Cellranger vdj function from Cellranger version 3.1.0 based on 10×Genomics provided hg 38/GRCh38 human reference genome (refdata-cellranger-vdj-GRCh38-alts-ensembl-2.0.0).

Analysis of scTCRseq: processed files by Cellranger were imported in R and clones without full-length and non-productive contigs (which compose the clonotypes of Cellranger) were discarded for downstream analysis. To improve the definition of clonotypes and considering that TRA and TRB chains are defined by combining CDR3 and V and C subunits, only those cells presenting only 1 TRA chain and 1 TRB chain were considered for analysis to define clones. To estimate clonal diversity, Gini-TCR Skewing Index⁴⁹ and Morisita's overlap index were used.

The clonotypes thus defined were then compared to different databases (McPAS-TCR, TBAdb and VDJdb) by accepting up to 1 amino acid mismatches on the TRB. The TRA was not considered. Cells with clonotype references in the databases were represented in UMAP according to three categories based on their associated Ag: Pathogenic or Autoimmune.

### ScATAC-seq Analysis

Pre-processing of scATACseq: demultiplexing and conversion of raw base call (BCL) files into Fastq files were performed using bcl2fastq2 version 2.20 (with the following options:—no-lane-splitting—minimum-trimmed-read-length=8—mask-short-adapter-reads=8—ignore-missing-positions—ignore-missing-controls—ignore-missing-filter—ignore-missing-bcls). To align and quantify scATAC-seq reads, and to generate fragment files (containing all unique scATAC-seq fragments linked to the corresponding cellID) for the different samples, cellranger-atac count from Cellranger ATAC version 1.0 was used with the hg38/GRCh38 human reference genome provided by 10×Genomics (refdata-cellranger-atac-GRCh38-1.0.0).

Analysis of scATACseq: Downstream analyses were processed using R package ArchR version 0.9.5-1.0.1⁵⁰ with R version 3.6.1. Annotation was done on hg38/GRCh38 human reference genome. Fragments files generated by Cellranger were imported in ArchR, low quality cells were discarded based on TSS enrichment (with a threshold fixed at 7) and fragments per cell (with a threshold fixed at 1,500). To resolve the technical differences observed in the lymph node sample from patient 2 compared to the other samples, down-sampling of the number of fragments was performed by taking only half of the fragments contained in this sample. Low quality cells of this sample were discarded as described above. Inferred doublets (using addDoubletScores function, a predictive approach proposed by ArchR which consists in the identification of nearest neighbor cells of in silico doublets from the data) were filtered after merge of all samples.

Elimination of contaminant cells: iterative Latent Semantic Indexing (iLSI), a dimensionality reduction method, was applied on data composed of all good quality cells from the different samples (using addIterativeLSI based on the “TileMatrix” with 10 iterations on 50,000 cells and 5,000 variable features and passed the following parameters to the addClusters function: resolution=0.1, sampleCells =50,000, n.start=10). Batch effect correction was performed using Harmony method. Processed data were clusterized using graph-based clusterization implemented in Seurat at different resolution (between 0 and 2 for each decimal) and UMAP reduction was computed to visualize the data. Contaminant cell clusters were defined using clustering with resolution 0.2, based on approximated gene expression scores (Gene Scores) (approach based on the global contribution of chromatin accessibility within the entire gene) of T cell markers (CD3D) and expression of other immune population markers (CD14, CD8A, CLEC4C or MS4A1).

Characterization of scATAC cell populations: identified contaminant cells were removed from the data and analysis was repeated. iLSI was performed on good quality data (using addIterativeLSI based on the “TileMatrix” with the same parameter as before). Batch effect correction was applied using Harmony method. Processed data were clusterized at different resolutions (between 0 and 2 for each decimal) and UMAP reduction was computed. Clusterization obtained at resolution 0.9 was used for downstream analysis. To characterize identity of cell clusters, the identification marker features proposed by ArchR was performed on matrix Gene Score (using Wilcoxon test and considering only genes with a minimum logFC of 0.2 and a maximum false discovery rate (FDR) of 0.05).

ScRNA-seq-scATAC-seq integration: to cross-compare cluster identities assigned using Gene Scores in scATAC-seq with identities characterized by gene expression in scRNAseq, Seurat's Transfer anchors process adapted by ArchR was performed with addGeneIntegrationMatrix function using 15,000 reference cells from both data sources. Resulting cluster identities and gene expression of scRNA-seq cells were reported to the closest scATACeq cells.

Peak calling: peaks calling was performed using MACS2 via the iterative overlap peak merging procedure proposed by ArchR (using addReproduciblePeakSet on cluster from resolution 0.9 with default parameters). Cisbp database was used to define and annotate peaks containing transcription factor (TF) motifs. Differential peaks among clusters were characterized using getMarkerFeatures function (using Wilcoxon test). From these differential peaks, first prediction transcription factor activity among clusters were performed (using peakAnnoEnrichment with a minimum logFC of 0.5 and a maximum FDR of 0.1) and visualized by heatmap (clusterized using euclidean distance with Ward's method). In a second time, ChromVAR deviation z-scores were calculated to predict TF enrichment for individual cells of the data. To predict the precise binding site of TF motif, ArchR's getFootprints function was used.

Peak signatures: public peak list signatures were used as peak annotation (using addPeakAnnotations). To convert genomic coordinates of peaks from mm10 mouse genome or previous (hg18/NCBI36, hg19/GRCh37) human genome to hg38/GRCh38 human reference genome, the web version of LiftOver tool from UCSC⁵¹ (https://genome.ucsc.edu/cgi-bin/hgLiftOver) was used. For peak signature obtained on previous human genome reference, a ‘minimum ratio of base that must remap’ of 0.95 was considered, while a ‘minimum ratio of base that must remap’ of 0.2 was considered as sufficient for signature characterized on mouse data. Using processed public signatures composed by liftover regions (resulting from LiftOver), signature enrichment (using peakAnnoEnrichment with a minimum logFC of 0.5 and a maximum FDR of 0.1, following by heatmap visualization using plotEnrichHeatmap) and calculation of ChromVAR deviation Z-scores were performed.

Visualization: MAGIC (Markov Affinity-Based Graph Imputation of Cells) smoothing signal procedure, imputing weight to each cell, was applied on gene Score, gene expression and deviation z-scores before visualization using UMAP reduction.

Co accessibility and Peak2Genes: co-accessibility between peaks of same gene among cells (co-accessibility) and co-accessibility between peaks and gene expression of same gene among cells (peak2genelinkage) were explored using ArchR's functions (using addCoAccessibility following by getCoAccessibility and addPeak2GeneLinks following by getPeak2GeneLinks with dimension data obtained with Harmony). Briefly, co-accessibility highlights peaks which accessibility correlates across many single cells; while peak2genelinkage highlights peaks which accessibility correlates with gene expression.

Regulatory network analysis: To construct regulatory network, scATAC-seq coupled to scRNA-seq were used. First, differential analysis was performed between naïve/memory, effector and TFH-like clusters to identify candidate target genes. In parallel, after selecting positive TFs based on Gene Integrated data, a binary matrix containing peaks scanned for these TF binding sites was extracted from data results obtained from ArchR (using getMatches). Then, the peaks contained in this matrix were linked to the union of candidate target genes using Peak2Genes output (using getPeak2GeneLinks function with the following parameters: corCutOff=0.5, resolution=10,000, returnLoops=FALSE). To further define the regulatory network, Pearson correlations between candidate target genes and TFs were performed on scRNA-seq data. Low and/or non-significant correlations (|Pearson correlation|>0.1 and p-value<0.001) and “TF-candidate target gene” pair without peak detection were removed. Finally, the regulatory network was designed from these results using Gephy.

Trajectory analysis: taken into consideration results from scRNA-seq trajectories and velocity analysis, supervised trajectory analysis in all tissues and per tissue (blood only, lymph node only and tumor only) were performed using ArchR's procedure. ArchR's supervised trajectory analysis determines a coordinate and a pseudotime value for each cell of the designed trajectory, based on distance between each cell and the next cluster in the trajectory. Once done, gene scores, gene expression, deviation z-scores and peak accessibility were visualized by heatmap and UMAP. Integrative analysis between correlated gene score and TF motif accessibility or between gene expression and TF motif accessibility were performed (using same parameters than ArchR manual) to produce paired heatmaps for the corresponding features.

Identification of Treg-FL precursors and resident Tregs and Tconvs by flow cytometry TDLNs from 3 patients with NSCLC and 2 tonsils from healthy donors were stained few hours after surgery with specific antibodies. Cell suspensions were washed in PBS and incubated with LIVE/DEAD Fixable Cell Dead Stain (eBioscience) during 15 min at RT. Next, cells were washed and incubated with fluorochrome labeled Abs for 20 min at 4 °C. For FOXP 3 intranuclear staining, cells were fixed, permeabilized and stained with Foxp3/Transcription Factor Staining Buffers (eBioscience) following eBioscience One-step protocol manufacturer's instructions. Data acquired with a BD LSR-Fortessa flow cytometer were compensated, exported into FlowJo software (version 10.0.8, TreeStar Inc.).

### Treg-FL Precursor Isolation.

Upon tissue dissociation, cells were washed in PBS and incubated with LIVE/DEAD Fixable Cell Dead Stain (eBioscience) during 15 min at RT. Next, cells were washed and incubated with fluorochrome labeled Abs for 20 min at 4 °C. Cells were FACS-sorted as CD 200high, BTLAhigh, ICOS+, PD1high from live CD4+CD25high cells from lymph nodes and tumors of two NSCLC patients (representative data of 7 patients) using BD ARIA Fussion flow cytometry.

### Genome Editing With Crispr-Cas9 Technology

Sorted Tregs and Tconvs were stimulated with IL-2 (300 IU/mL) and with soluble αCD3αCD28αCD2 (25 μL/mL) 24 hours before genome edition. Two IRF1 CRISPR RNA (crRNA) guides were designed to target the exon 2 and 3 of IRF1 with Integrated DNA Technologies tools (IDT). The two IRF1 crRNAs were mixed independently to trans-activating crRNA (tracrRNA, IDT) at an equimolar ratio and incubated 30 minutes in a 37 °C-5% CO2 incubator to form two single-guide RNAs (sgRNAs) at 100 μM. HiFi-Cas9 protein (IDT) was mixed with each sgRNA independently at a ratio 2sgRNA: 1Cas9 to form two CRISPR ribonucleoprotein complexes (crRNPs) at a concentration of 50 μM. The crRNPs were incubated 10 minutes at room temperature (RT). Tconvs and Tregs were pulled down by centrifugation at 350 rcf for 10 min at RT, media was removed, and 0.5-2.106 cells were resuspended in 20 μL of P3 Primary Cell Nucleofector Solution (Lonza). 2.5 μL of each crRNP (50 μM) were added to the 20 μL of cell suspension (total of 25 μL) and the mix was incubated at RT for 2 minutes. Cells with crRNA were then transferred into 16-well Nucleocuvettes (Lonza) and electroporated using an EH100 pulse with Lonza, 4D-Nucleofector Core. Four days after the electroporation, the efficacy of the gene deletion was assessed by Western blot (WB) and FACS and the electroporated cells were reactivated with IL2 (300 IU/mL) and αCD3αCD 28 coated beads using a ratio 1bead: 1cell for further expansion.

### Cell Stimulation for IRF1 Expression

1.105 cells were stimulated in 200 uL of completed Xvivo with 1000 IU/mL of IFNγ (Miltenyi) in round-bottom 96-well plates. They were stimulated during 24 hours prior to evaluation by flow cytometry, or during 6 hours prior to RNA sequencing. For cytokine production assessment, 2.105 cells were stimulated in 200 uL of completed Xvivo with Cell Activation Cocktail (Biolegend) in round-bottom 96-well plates. They were subsequently stained, fixed, and analysed using multi-parametric flow cytometry.

### Western Blot Analysis

Treg and Tconv cells were lysed in RIPA buffer (150 mM NaCl, 20 mM Tris pH=8, 0.5 mM EDTA in milliQ water) supplemented with protease inhibitor (1:50, Sigma) and benzonase (1: 200, Sigma) 4 days after genetic edition. Cell lysates were separated alongside SeeBlue Plus2 Prestained standard protein ladder (Fisher) in a Blot 4-12% Bis-Tris Gel (Biorad) and transferred onto PVDF membranes. Membranes were probed overnight with α-IRF1 (1:1000; D5E4; Ozyme) and α-actin (1:9000; A2228, Sigma-Aldrich) primary antibodies. Membranes were then incubated with HRP-conjugated anti-mouse IgG (71045, EMD Millipore) and anti-rabbit IgG (sc-2357, Santa Cruz Biotechnologies) secondary antibodies (1:10000). Antibody binding was detected by chemo luminescence using ECL substrate (Fisher), and the signal was visualized with a Chemidoc Imager (BioRad).

### Generation of Tumor Model in Humanized Mice and Transfer of Human Cells

Nod Scid Gamma (NSG) mice were bred, housed, and fed with autoclaved food and water in the OPS from Curie Institute facility. After 7 days of adaptation, mice were subcutaneously injected on the flank with 100 μL of PBS containing 5.10⁶ of human breast cancer MDA-MB 231 cells (day 0). When the tumor was palpable (between day 6 and day 10), mice were injected intravenously with 100 μL of PBS containing four human-cell preparations with 3 mice per group. PBMCs were recovered from HLA-A2(+) donors, while Treg and Tconvs were from HLA-A2(−) donors. Total PBMCs were stained to evaluate the percentage of CD3⁺ cells. The four different PBMC+ Treg/Tconv mixes were composed of 100 μL containing 5.10⁶ CD 3⁺cells (PBMC) and: 1-5.10⁶ WT Tregs; 2-5.10⁶ IRF1 KO Tregs; 3-5.10⁶ WT Tconvs; 4-IRF1 KO Tconvs. 6 days after cell transfer, liver, spleen, and tumor were collected and processed to recover cell suspension for phenotypic characterization. Spleen was smashed, the recovered cell suspension was filtered and lysed with red-blood cell lysis buffer.

Liver was smashed, the recovered cell suspension was filtered and processed with a percoll gradient to remove debris. Tumor was weighted, cut into pieces, digested with liberase (60 μL/mL, Sigma) and DNAse (30 μL/mL, Sigma) and mechanically digested using the AutoMACS device (MILTENYI) and the recovered cell suspension was filtered. For the three tissues, cells were counted and stained to evaluate the phenotype and infiltration of WT and IRF1 KO Tregs and Tconvs by flow cytometry.

### Results

### scRNA-seq Reveals Multiple Pure Treg, Pure Tconv, and Mixed Treg/Tconv Subsets Among CD4+ T Cells From NSCLC Patients

To assess the transcriptomic landscape of CD4+ T cell in NSCLC patients, scRNA-seq coupled to TCR-seq was performed in CD4+ T cells isolated from matched tumor, metastatic LNs and blood of treatment-naïve patients. Tregs and Tconvs from each location were FACS-sorted as CD 25^{high}CD127^{low} and CD25^{low}CD127^{high/middle}CD45+CD4+ live cells, respectively. As Tregs are typically less abundant than Tconvs, these populations were mixed at a 1:1 Treg/Tconv ratio for sequencing to increase the power for characterizing Treg subpopulations (data not shown).

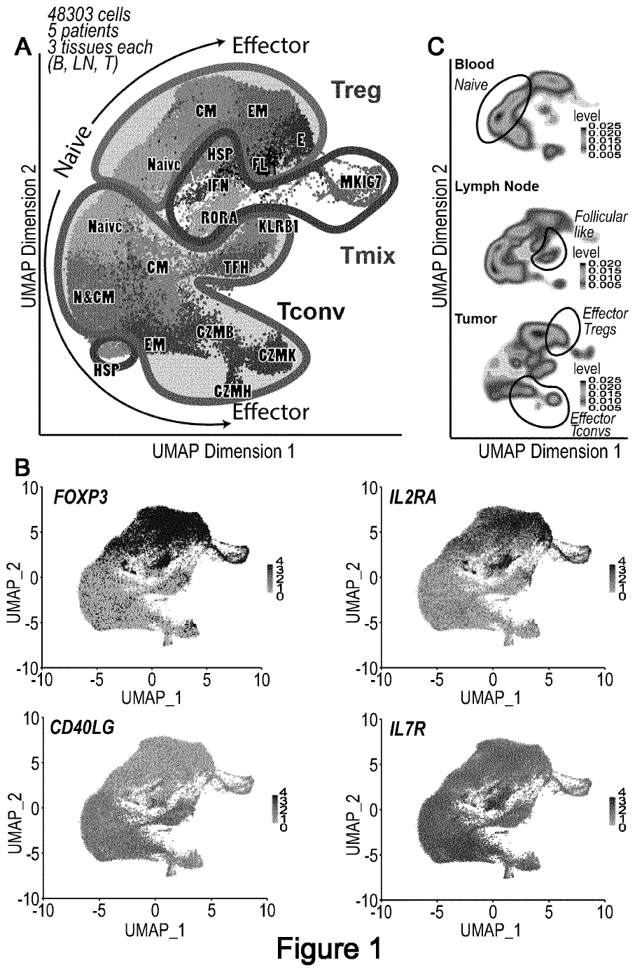

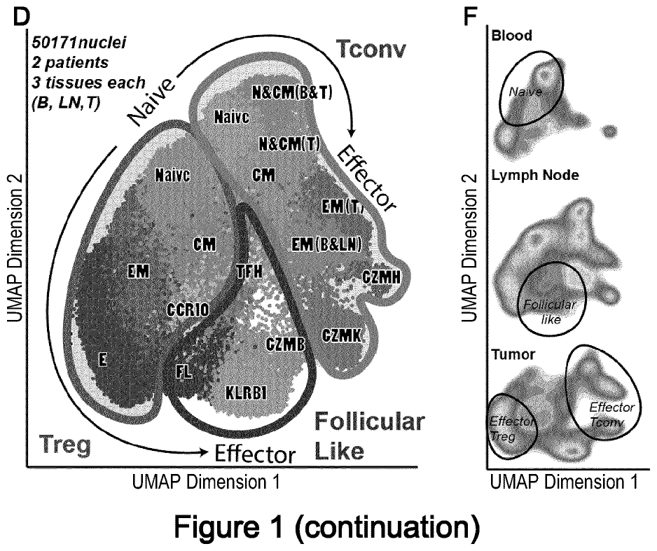

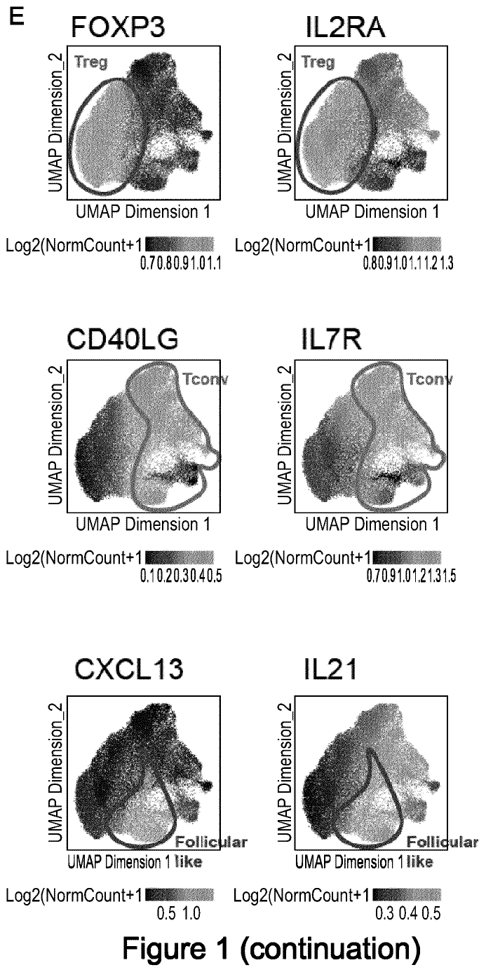

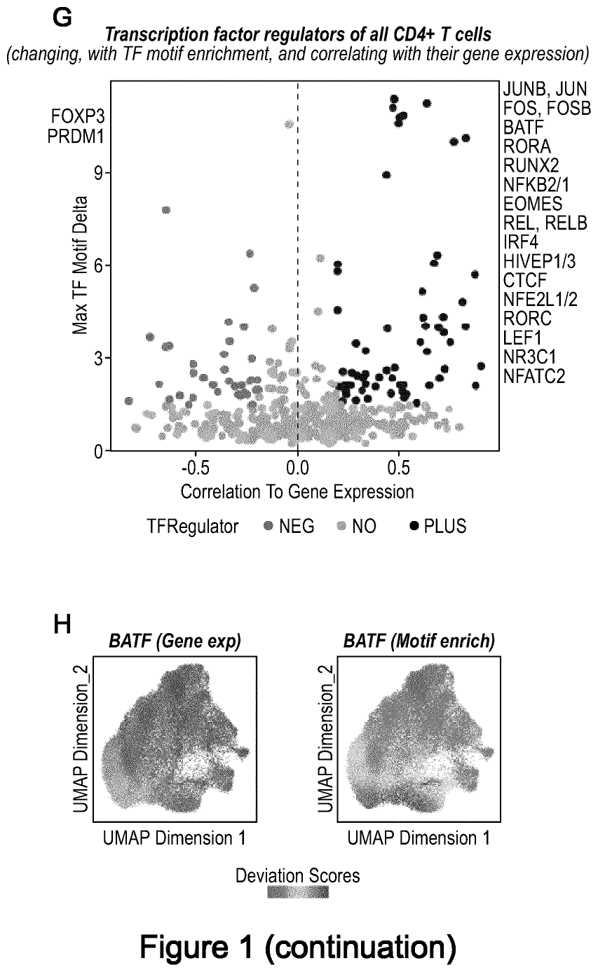

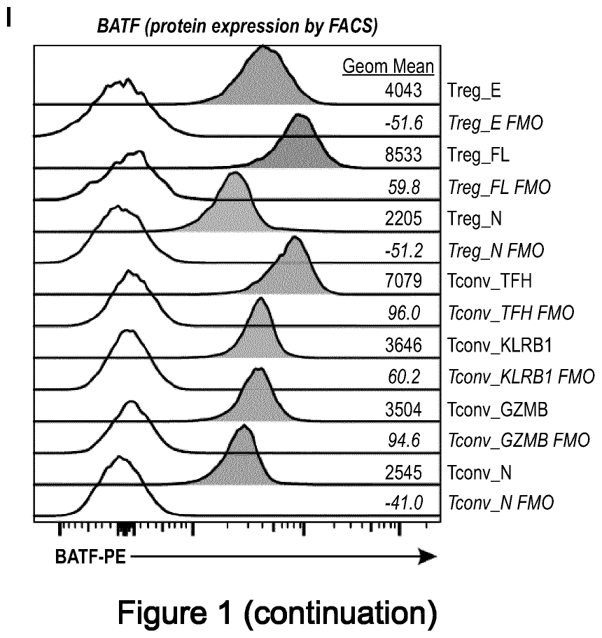

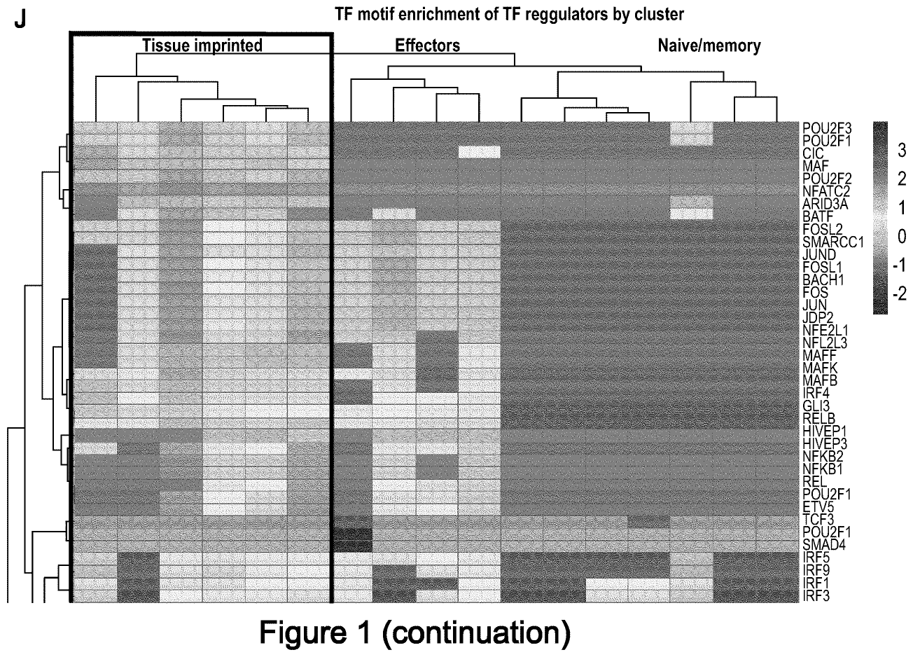

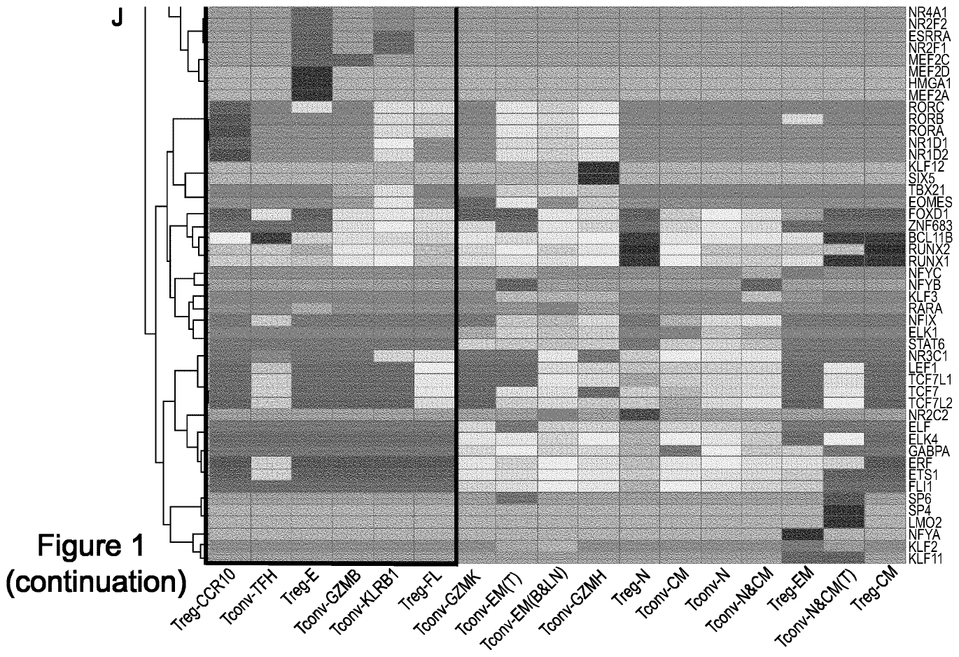

For the transcriptomic/TCR analysis the CD4+ T cells from five individuals was studied (three tissues in each patient, for a total of 15 samples) (data not shown)). The transcriptome of 48,383 cells was recovered in total after quality control (data not shown). Cells from all patients and tissues were integrated in a single dataset. Using unsupervised graph-based clustering 21 clusters were identified. Uniform manifold approximation and projection (UMAP) visualization and quantification of cells by cluster are shown in FIG. 1A. The relative proportions of these subpopulations varied highly across tissues, but the majority of clusters were represented in all the patients (data not shown).

Based on the analysis of clustering resolution hierarchy (data not shown), differentially expressed genes (data not shown), and pathognomonic gene and signature expression (FIG. 1B and data not shown) cell clusters were classified as “pure” Tregs, “pure” Tconvs or “mixed” Treg/Tconv (Tmix). The five pure Treg clusters expressed high levels of FOXP3 and IL2RA transcripts and were enriched in Treg signatures. The nine pure Tconv clusters expressed high levels of IL7R, CD40LG and THEMIS transcripts. The four Tmix clusters presented a mix of cells with characteristics of Tregs and Tconvs, but their transcriptomic signatures were driven by cell states such as “cycling”. In more detail, the five Treg clusters segregated as: naïve (Treg-N; expressing LEF1 and CCR7), central memory (Treg-CM; SELL and S100A4), effector memory (Treg-EM; HLA-DR and CXCR3), effector (Treg-E; CCR8, TNFRSF9, and ILIR2), and follicular-like Tregs (Treg-FL; CXCL13, ICA1, and ILIR2). The nine Tconv clusters were identified as: naïve (Tconv-N; TCF7, SELL and RPS6), naïve & central memory (Tconv-N&CM; S1PR1 , ANXA1, and GPR183), central memory (Tconv-CM; S100A4, AREG, TCF7), effector memory (Tconv-EM; ANXA1, and NFKBIA), T follicular helper (Tconv-TFH; ICOS, CXCL13, and BCL6), three different effector populations (Tconv-GZMB,-GZMH, and -GZMK), and pro-inflammatory/exhausted T cells (Tconv-KLRB1; KLRB1, TOX, and IL17A). The main four Tmix clusters were identified as: cycling (Tmix-MK167; MKI67, TOP2A), activated (Tmix-RORA; RORA, STAT3), IFN response (Tmix-IFN; IFIT1, ISG15), and stress response (Tmix-HSP; HSPE1, DNAJBI). Three Tmix clusters were excluded from the characterization due to low cell counts, to TCR bias or to being contributed by only one patient. Each cluster has been characterized in detail (data not shown)

Exploration of the tissue source of the cells showed that not all clusters were equally represented in each tissue. Quantification of frequencies of cells per cluster in the three tissues (heatmap) and of observed vs expected proportions (bar plots) (data not shown), revealed that naïve and CM Tregs and Tconvs were enriched in the blood and LNs; Tconv-TFH and Treg-FL clusters were enriched in the LNs and tumor; and the mix clusters HSP and RORA, as well as the finally differentiated effector T cells were enriched in the tumor (UMAP density plots, FIG. 1C and data not shown). Overall, scRNA-seq analysis of a large number of CD4+ T cells purified from blood, LNs and tumors from NSCLC patients revealed a high diversity of CD4+T lymphocytes with distinct transcriptomic characteristics and differential distribution among tissues.

### Treg, Tconv, and Tfh-Like Epigenetic Programs Define the Cd4+ T Cell Landscape in NSCLC

scATAC-seq of CD4+ T cells from two NSCLC patients was performed using the same sorting strategy as for scRNA/TCRseq (data not shown). The chromatin accessibility landscape of a total of 50,171 cells passing the quality controls (data not shown) was generated. Each nucleus yielded on average of 11.5×10³ unique fragments mapping to the nuclear genome, and a fraction of 0.6 reads in peak regions. Around 7,317 peaks/cell and 8,347 predicted genes/cells (gene expression estimated on the accessibility of regulatory elements in the vicinity of the gene, gene score) were obtained, compared to the 1,260 genes/cell obtained by scRNA-seq.

As with the scRNA-seq, all scATAC-seq samples were first integrated into one dataset and used unsupervised graph-based clustering to define 17 accessibility subsets. FIG. 1D shows the distribution of nuclei by cluster and by tissue. Unsupervised hierarchical clustering based on gene score and chromatin accessibility of cis-acting DNA elements (i.e. ATAC-seq peaks of enhancers and promoters), (data not shown), assigned the 17 subsets into three main groups: Tregs, Tconvs and a third group with characteristics of follicular T cells (Follicular-like), distinguished respectively by the canonical markers FOXP3 and IL2RA; CD40LG and IL7R; as well as CXCL13 and IL21 (FIG. 1E).

In more detail, Treg clusters included: naïve (Treg-N; LEF1 and CCR7), central memory (Treg-CM; SELL and AREG), effector memory (Treg-EM; HLA-DR and TNFRSF4), effector (Treg-E; TNFRSF9, CCR8, CD80), and a minor CCR10 group. Tconvs clusters comprised: naïve (Tconv-N; SELP, NOSIP and SELL), central memory (Tconv-CM; AREG, and ANXA1), two naïve & central memory clusters, one enriched in blood and LNs and one enriched in the tumor (Tconv-N&CM(B&LN) and Tconv-N&CM(T); S1PR1 , and TCF7), two effector memory clusters (Tconv-EM(B&LN) and Tconv-EM(T); ANXA1, NFKBIA, and TNF), and two effector Tconv clusters (Tconv-GZMH; GZMH, and GNLY ; Tconv-GZMK; GZMK, and EOMES). Follicular-like clusters contained follicular-like Tregs (Treg-FL; TCF7, ILIR2, and ICAI), follicular Tconvs (Tconv-TFH, CXCR5, BCL6, and CXCL13), pro-inflammatory/exhausted Tconvs (Tconv-KLRB1; KLRB1, TOX, and IL17A) and tissue resident T convs (Tconv-GZMB; GZMB, CCL3, and ITGAE). Extensive characterization of each cluster was performed (data not shown).

To understand the relationship between the transcriptomic and the chromatin characteristics of each cell cluster, the scATAC-seq and scRNA-seq datasets were integrated and the scATAC-seq clusters were labelled with scRNA-seq cluster annotations (data not shown). It was observed that “pure Treg” and “pure Tconv” clusters were conserved between expression and accessibility maps, but the T-mix clusters defined at the transcriptomic level were not identified as distinct clusters at the chromatin level, implying that cell states such as cycling and interferon response greatly alter transcriptome profiles while affecting global chromatin profiles much less dramatically.

Overall, integration of scRNA-seq with scATAC-seq profiles, where transient cell states are less prominent, induces a rearrangement in the cluster architecture. In particular, the integrated grouping highlights a set of Treg and Tconv cells sharing a follicular program. In order to understand this molecular program, the underlying patterns of transcription factors and their target genes were next investigated.

### scATAC-seq Uncovers a Common Gene Regulatory Network Imprinting Tissue Residency

To identify transcription factors (TFs) potentially controlling the gene expression program of each cell cluster, the integrated RNA/ATAC atlas generated by the inventors was leveraged to select TFs whose gene expression was positively correlated to changes in the accessibility of their corresponding binding motifs (TFBM, TF-binding motifs), and performed unsupervised clustering (FIG. 1G-J). The strongest drivers of the different clusters identified through this approach included JUN/JUNB, FOS/FOSB, BATF (FIG. 1H), RORA, RUNX2, MAF, and IRF4 (data not shown). Of note, the gene expression of FOXP3 and PRDMI (BLIMP-1) were negatively correlated with the accessibility of their TFBS (i.e., in cells expressing FOXP3 the chromatin binding sites for this TF are less accessible), consistent with their known function as transcriptional repressors. The most differentiated effector cells showed molecular programs characterized by the high and specific activity of SMAD4, TCF3, and ETV5 (in Tconv-GZMK); and MEF2A-D and HMGAI (in effector Tregs) (data not shown).

Unsupervised clustering using the above selected TFs classified the CD4+ T cells into three main TF-driven groups. One group included the naïve/memory Tconv and Treg clusters; a second group included Tconv effectors, and a third group included Treg-E and the Follicular like cluster. Similar results were obtained when applying unsupervised hierarchical clustering using chromatin accessibility of cis-acting DNA elements (FIG. 1J).

Among candidate active TFs identified by this approach, the naïve/memory program was regulated by TCF7 and LEF111; the effector phenotype was guided by KLF2, EOMES, TBX21, RORA, and RORC¹²; and the third group showed a strong enrichment for BATF⁹, POU2F1-3, MAF and NFATC2¹³ (data not shown). Given that (i) BATF has been identified as a key TF determining the molecular program of tissue residency for Tregs^{9,14} and CD8+ memory cells, (ii) this third group of clusters specifically expresses molecules associated to tissue residency, such as CXCR3, CXCR6, CD69, ILIRLI and PRDM1¹⁵ (data not shown), (iii) this group does not express known determinants of T cell circulation and tissue egress such as CCR7, SELL, S1PR1 , and KLF2 (data not shown), and (iv) this group is mainly found in both LNs and tumors, but not in blood (data not shown); this group was called tissue-imprinted. Additionally, the effector and tissue-imprinted cell subpopulations shared a common program of activation characterized by the JUN, FOS, REL, HIVEP1-3, NFE2L, and MAF family members¹² (FIG. 1C, F).

To verify at the protein level that BATF expression is characteristic of the five tissue-imprinted clusters, a FACS gating strategy was first designed allowing the identification of these five populations (FIG. 1I). Application of this strategy to blood, LN, juxta-tumor and tumor samples from NSCLC patients confirmed that Treg-FL and Tconv-TFH cells are absent from blood and juxta-tumor tissues; while Treg-E, Tconv-GZMB, and Tconv-KLRB1 cells are found at higher levels in the tumor tissue. As expected, BATF protein was more highly expressed in the tissue-imprinted subpopulations compared to naïve Tconv and naïve Treg cells (data not shown). Additionally, MAF protein levels followed a similar expression pattern, validating the inferred results obtained by these multiomics approach (data not shown).

To further study the tissue-imprinted program shared by the Folicular-like clusters and the Treg-E, their TF: target gene regulatory network was constructed as previously described16. the correlation between the transcripts of the DEGs of each of the 5 populations and their positive TF-regulators was calculated; and filtered for the genes with accessible TFBM (identified by peak-to-gene linkage analysis, data not shown). To build the TF: target networks, the most connected TF-regulators and target genes were kept (data not shown). All tissue-imprinted populations shared a subset of common regulators, underlying T cell activation (JUN/B/D, FOS/B) and tissue residency (BATF); but these were organized in distinct TF-target genes architectures, reinforcing each self-type identity. Tconv associated TFs included NFATC2, NR3CI and RUNX2; and Treg associated TFs included IRF1, NFKB2 and RELB, while Tconv-KLRB1 cells expressed a mix of them. Interestingly, BATF which is central to the tissue imprinted signature, is predicted to regulate the expression of different sets of genes in each cell type. For example, among the Tconvs, BATF regulates the differential expression of CXCL13 in Tconv-TFH; CD69 and CD44 in Tconv-GZMB; and IFN-g and TOX2 in Tconv-KLRB1. Conversely, in Tregs, BATF is associated with a different group of genes, including IL-21R, SOCS1, NFKB2, in the two Treg populations; ZNF281 in Treg-FL, and with CCR8 and TNFRSF9 in Treg-E (data not shown).

Overall, combined scATAC-seq and scRNA-seq revealed regulatory programs characteristic of the different CD4+ T cell clusters and identified a tissue residency program shared by a group of activated Tregs and Tconvs. Tissue-imprinting was dictated by both known and newly identified TFs, that organized in unique network architectures self-reinforcing the identity of these five cell types.

### Tcr Sequencing Reveals the Phenotypic Landscape of Clonal Expansions Across Tissues

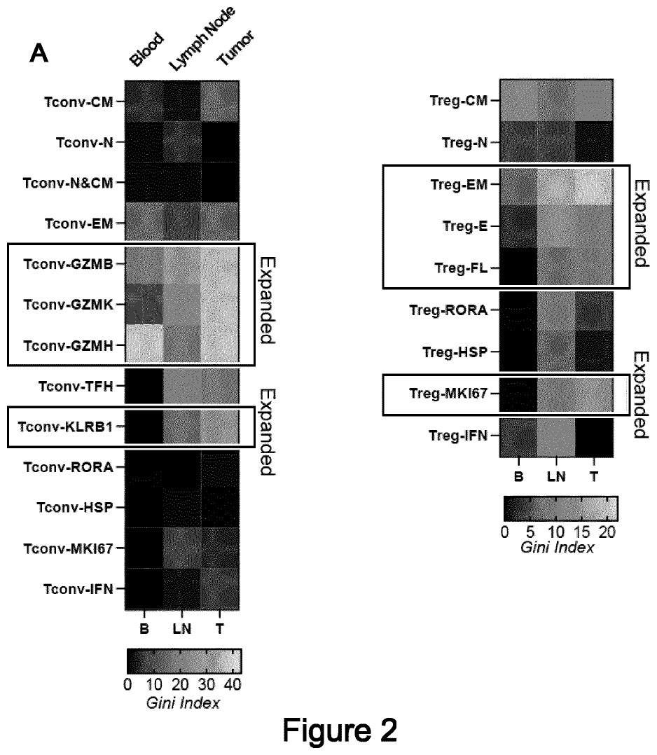

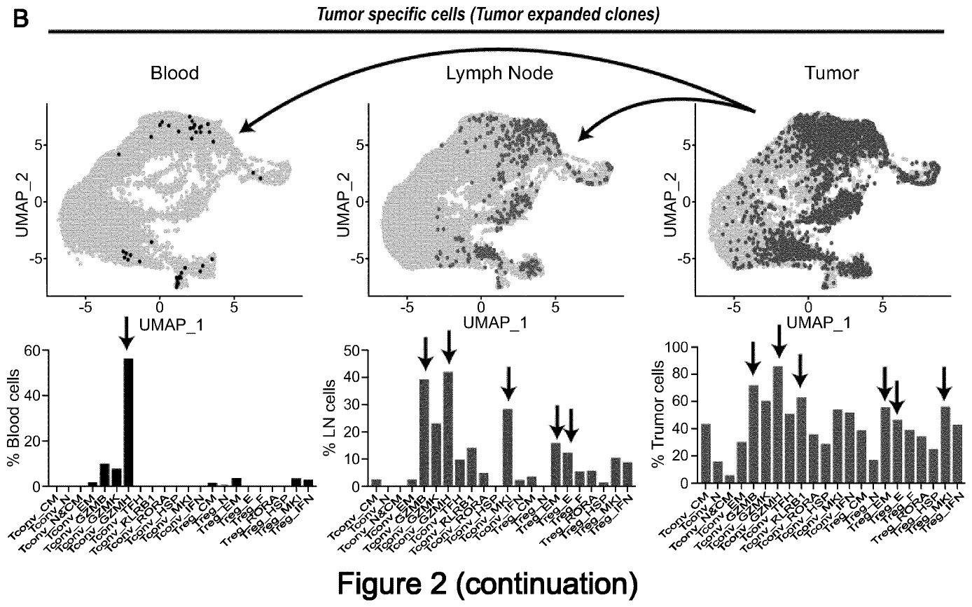

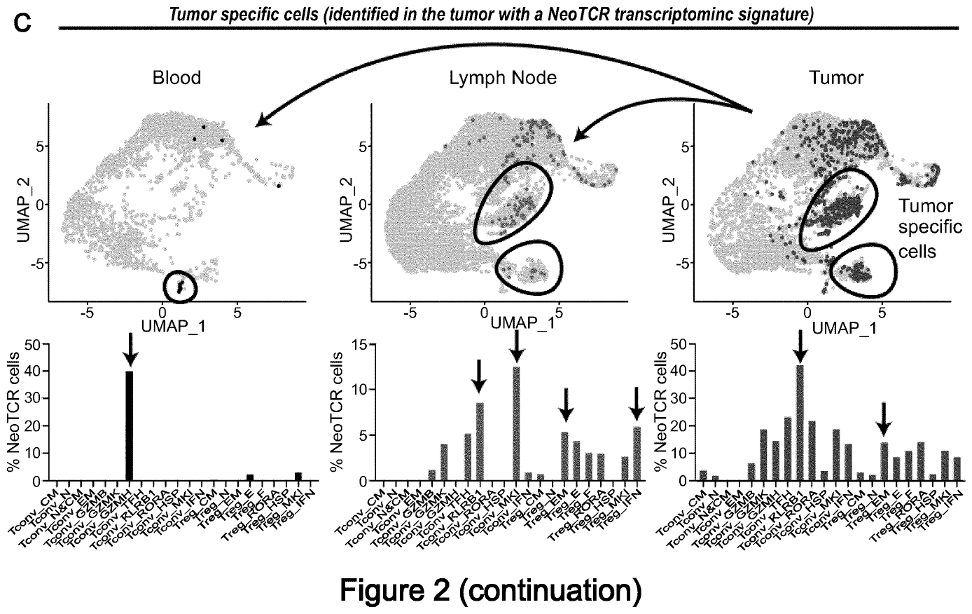

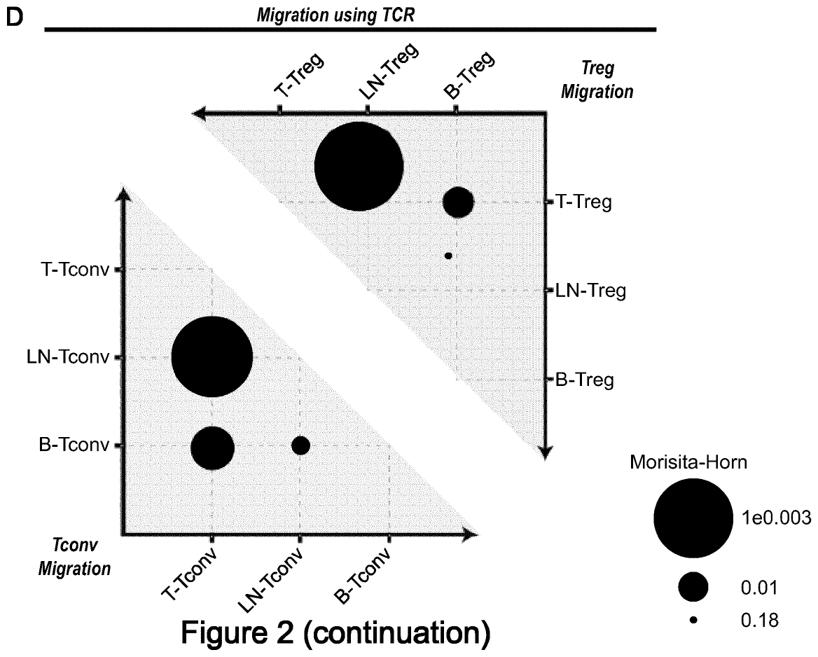

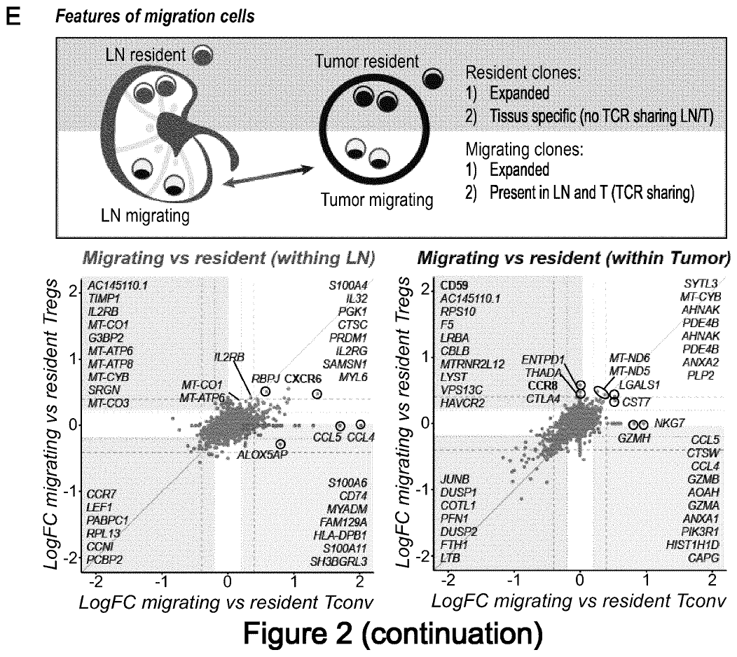

To investigate the clonal relationship among the different CD4+ T cell subsets in the three tissues, the transcriptome and paired TCRa/B sequences from three patients were integrated. A total of 16,865 cells, corresponding to 12,810 different clonotypes were obtained (data not shown). Cells from expanded clones (two or more cells expressing the same a/B TCR) were localized in discrete zones of the UMAP, which differed between blood and tissues (data not shown). To gain more insight into the clonal architecture of the different clusters, cells belonging to the Tmix clusters were computationally re-assigned into Tregs or Tconvs based on their expression similarities (data not shown). Next, the frequency (pie charts) and the absolute numbers (bar plots) of individual cells was plotted according to clonal abundance (data not shown); and clonal expansion was quantified by the Gini index for each patient and tissue (FIG. 2A). In all tissues, the vast majority of clones were unexpanded singletons. In the blood 29 % of cells came from expanded clones; the lowest TCR diversity was observed in the effector Tconvs (mainly in Tconv-GZMH), and in the memory Tregs. As expected, circulating naïve cells were the most polyclonal. In the LNs and tumor, 31 % and 52 % of all cells were from expanded clones, respectively. In both tissues, higher clonal expansion was observed among effector and effector-memory Tconvs and Tregs. However, in the tumor, Tconvs were more expanded than in the LNs, and presented with a final effector phenotype (mainly-GZMH,-GZMB,-GZMK) and KLRB. Additionally, for Tregs, it was observed that expanded clonotypes were found across all non-naïve clusters in the LNs, and more restricted to memory and effector phenotypes in the tumor (data not shown). Overall, these results support that cells undergo activation and clonal division within both the LNs and the tumor.

To investigate whether tumor-expanded clones-likely recognizing tumor antigens (Ags) were present in other tissues, their TCRs was used as lineage barcodes and tracked their phenotypic characteristics among the three tissues. Seven % of all expanded clones in the tumor (200 cells from 63 clones) were identified in the blood, with a strong enrichment in the GZMH cluster. Additionally, 50 % of expanded clones in the tumor (2,019 cells from 450 clones) were identified in the LNs, with the highest enrichment in the GZMB, GZMH, KLRB1, Treg-E, and Treg-EM clusters (FIG. 2B). To investigate potential Ag specificities of the clonally expanded TILs, CD4+CDR3beta TCR databases were interrogated public with annotated antigen targets. It was found that out of the 3,411 tumor-clonally expanded T cells from 901 clones, 74 cells from 24 clones expressed annotated pathogen-associated TCRs, and 84 cells from 23 clones expressed TCRs previously identified in autoimmunity. These few cells were observed across the different cellular states and tissues without a defined pattern (data not shown). It has been recently described that tumor Neo-Ag specific CD4+ TILs can be identified by a specific transcriptomic signature¹⁷. Using this approach, it was observed that in the tumor, 735 cells from 257 clones were classified as Neo-Ag specific T cells (FIG. 2C). This Neo-Ag specific signature was enriched among CD4+ Tconv cells in the Tconv-KLRB1cluster and found in lower proportions among the Tconv-TFH and Tconv-RORA states, suggesting an ongoing Tconv activation and expansion in tertiary lymphoid structures (TLS). In the LNs, these clones were also present among Tconv-KLRB1 and Tconv-TFH cells, and the highest proportion was detected among cycling cells, indicating that tumor-specific T cells that are primed in the LNs actively proliferate and acquire a follicular-like program. In blood, cells from clones expressing the Neo-Ag specific signature in the tumor were only found in the small Tconv-GZMH cluster. Although this Neo-Ag characteristic expression signature could also be detected in Treg cells, the interpretation is more difficult, as this signature has not been experimentally validated for Tregs¹⁷.

Overall, these results suggest that the landscape of TCR expansion is similar in metastatic lymph nodes and tumors, while in the blood many fewer expanded clones are found and these primarily express the Tconv-GZMH phenotype. Of note, tumor-expanded effector (Tconv-GZMK and GZMH) and follicular-like (Tconv-KLRB and Tconv-GZMB) Tconvs, which highly recirculate between the tumor and LNs, likely bear different TCR specificities: while the follicular-like cells seem to recognize tumor NeoAgs. The Ags recognized by the effector Tconvs remain unknown, although they are not associated to pathogens or autoimmune diseases in current databases.

### Shared TCRs Reveal Patterns of Migration and State Transitions

TCR sharing between cells does not only inform on cell migration (inferred from cells with the same TCR found in different tissues), but also on the transition of cells through different states (inferred from cells sharing the same TCR and being present in different expression clusters), as observed upon T cell activation and differentiation to effectors¹⁸. First, the migration of Tregs and Tconvs between the different tissues was studied. For this study Tregs and Tconvs were considered as whole populations (instead of by fine-grained clusters). all Treg and all Tconv pure and mix clusters from each tissue were pooled and computed the level of TCR sharing using the Normalized Morisita-Horn index (MHI: 0=no TCR sharing, 1=100 % sharing) (FIG. 2D). Tregs and Tconvs were each found to migrate mainly between LNs and tumors (0.19 MHI, 299 shared clones for Tregs and 0.18 MHI, 329 shared clones for Tconvs).

To compare the characteristics of migrating versus locally expanded cells, differential gene expression analysis of the migrating/resident Tregs vs migrating/resident Tconvs (Fc/Fc) was performed and Ingenuity Pathway Analysis (IPA) was applied (FIG. 2E and data not shown). The transcriptional program shared by migrating Tregs and Tconvs in the LNs, indicate that cells activate, polarize, extravasate, and use CXCR6 for migration¹⁹, relying on a glycolytic metabolic pathway²⁰. In the tumor, both cell types adapt to the hypoxia and nutrient-deprived milieu, as reflected by the activation of the oxidative phosphorylation, mitochondrial activity and response to TEM-related stimuli, such as sirtuin, estrogen and glucocorticoids. Unique to migrating Tregs in the tumor is the upregulation of CCR8²¹, CD59²² and ENTPD1 (CD39). These results put in evidence both shared and unique cues of CD4+ Tconvs and Tregs in LNs and tumor, which could be therapeutically modulated to selectively modulate Tconv or Treg migration and function.

To understand in more detail the gene expression dynamics of migrating cells the analysis was separated by cluster and tissue (data not shown). Overall, most of the migrating clones conserved their expression identity while trafficking between LNs and tumor (circles in the diagonal, data not shown), and transitions were mostly observed among Tregs, reflecting their higher tissue adaptation. In the LNs, TCRs also present in the tumor were enriched in the effector Treg and Tconv clusters (data not shown). In the tumor, cells with shared TCR with the LNs were present in the majority of the clusters (except naïve and central memory), and cells with shared TCR with the blood were mainly in the GZMH cluster (data not shown).

To better understand whether as T cells proliferate, they conserve their cluster identity or instead transit through different states, TCR sharing among expanded clones present in the LNs or in the tumor was analyzed using Normalized Morisita-Horn index. In the LNs (data not shown), out of the 9.7 % of expanded clones, 25 % were found at the same time in different clusters, while the majority of the expanding cells kept the same transcriptomic identity. Within Tconvs, the TCRs were shared among Tconv-GZMB clones and Tconv-MKI67 or Tconv-RORA clusters, in accordance with activation and proliferation of this highly expanded cluster. Within Tregs, Treg-N, Treg-CM and Treg-EM shared their TCRs with cells in the IFN-response and HSP mix clusters, while Treg-FL clones shared their TCRs with Treg-E. These results indicate that active Treg and Tconv cell cycling in the LNs is associated with transcriptomic changes of state.

Additionally, out of the total expanded clones in the LNs, 11.6% showed Treg/Tconv TCR sharing. The top Treg cluster sharing TCRs with Tconvs was the Treg-MKI67 that highly shared clones with final effector Tconv cells, resonating with the transient FOXP3 expression described during CD4+ Tconv activation²³ (upper circos plot insert in data not shown). For Tconvs, Tconv-KLRB was the top cluster sharing TCRs with Treg cells present in the CM, FL or E clusters (bottom circos plot insert in data not shown), likely underlying T cell activation in lymphoid follicles. Besides, some clones with cells present in both Treg and Tconv clusters were found among the different mix states, probably reflecting transient transcriptomic changes in cells undergoing TCR activation. To understand which was the fate of the clones that share TCRs among Tregs and Tconvs when reaching the tumor, the distribution of representative clones was visualized in UMAP plots (data not shown). The obtained results reinforce the notion that Treg/Tconv transiting cells in the LNs, reach a final effector Tconv phenotype in the tumors.

In the tumor, out of the 19.7 % of total expanded clones, 24 % were found at the same time in different clusters (data not shown). Compared to the LNs, more Tconv clones were found to be changing states: the highest TCR sharing was observed between Tconv-TFH and Tconv-KLRB1 clones, likely underlying reactivation in tertiary lymphoid structures. Additionally, high TCR sharing was observed among Tconv-CM, and Tconv-EM or Tconv-GZMB, likely recapitulating T-cell reactivation after arriving from the LNs. Moreover, clones from the Tconv-CM, Tconv-EM, Tconv-GZMB, Tconv-GZMK and Tconv-KLRB1 clusters were also found transiting in the IFN-response and HSP mix states. Finally, Treg-CM and Treg-EM transited through different states (IFN, MKI67, HSP) on their way towards final effector activation.

At odds with the LNs, Treg/Tconv sharing among the mix states were rare in the tumor. However, the top Treg and Tconv clusters sharing TCRs with their counterparts followed a similar pattern as in the LNs (circos plots in data not shown).

These results point out that CD4+ T cells present in the tumor and in its draining LNs, are found throughout different levels of activation and transiting across different states, underlying ongoing immune responses which, overall, reach a more terminal state in the tumor,

### Splicing Dynamics Uncovers a Novel Developmental Pathway of Tissue-Imprinted Cells

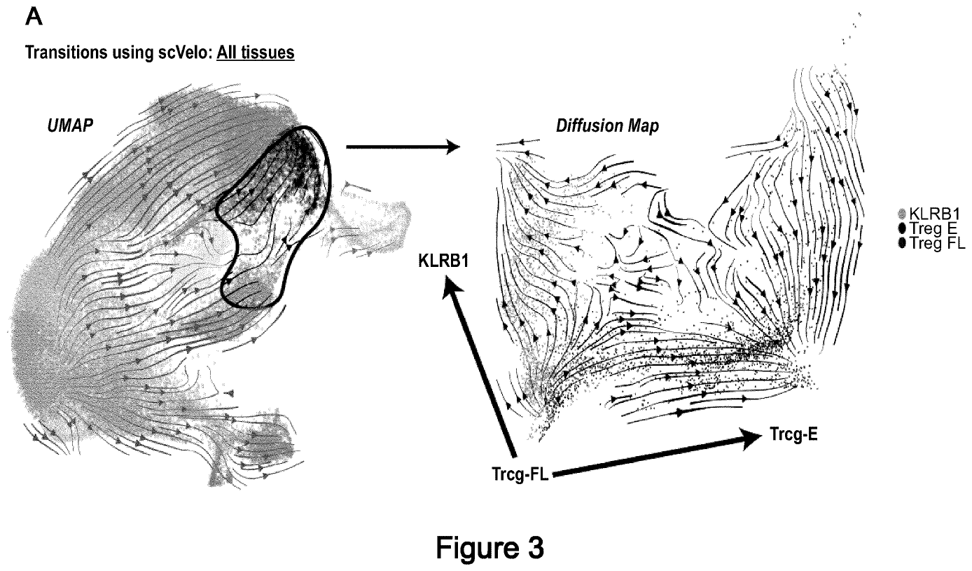

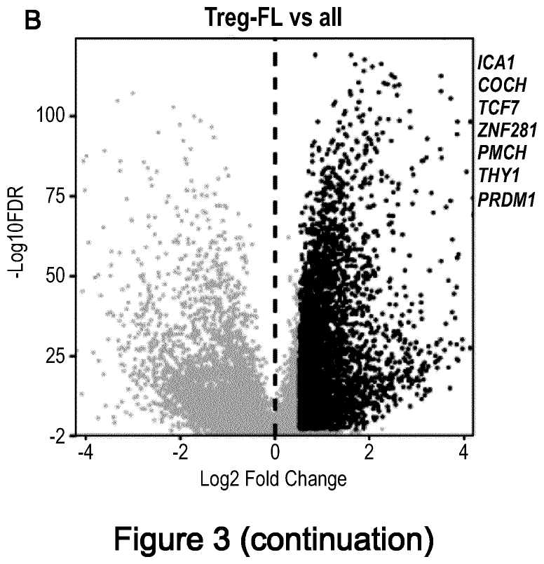

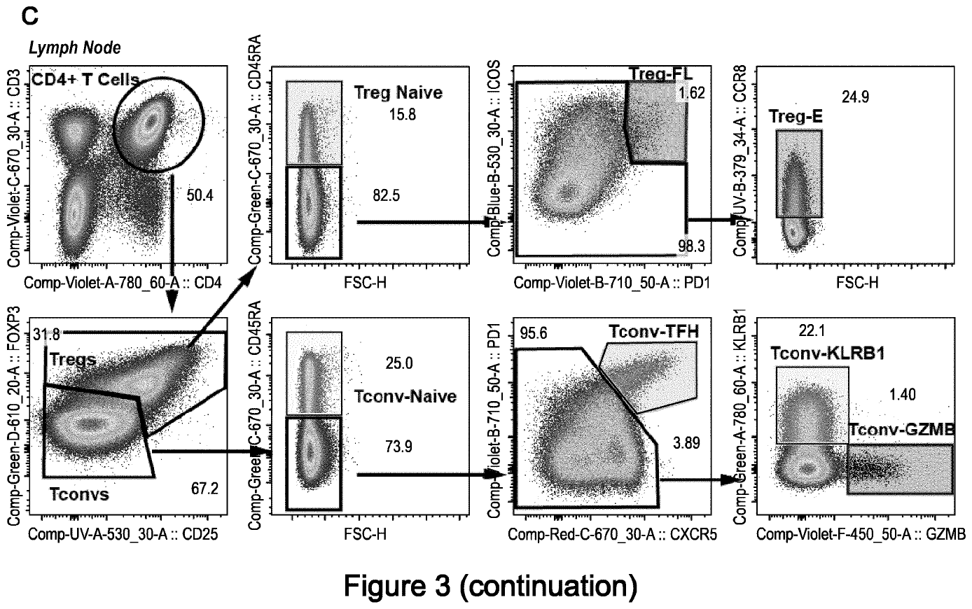

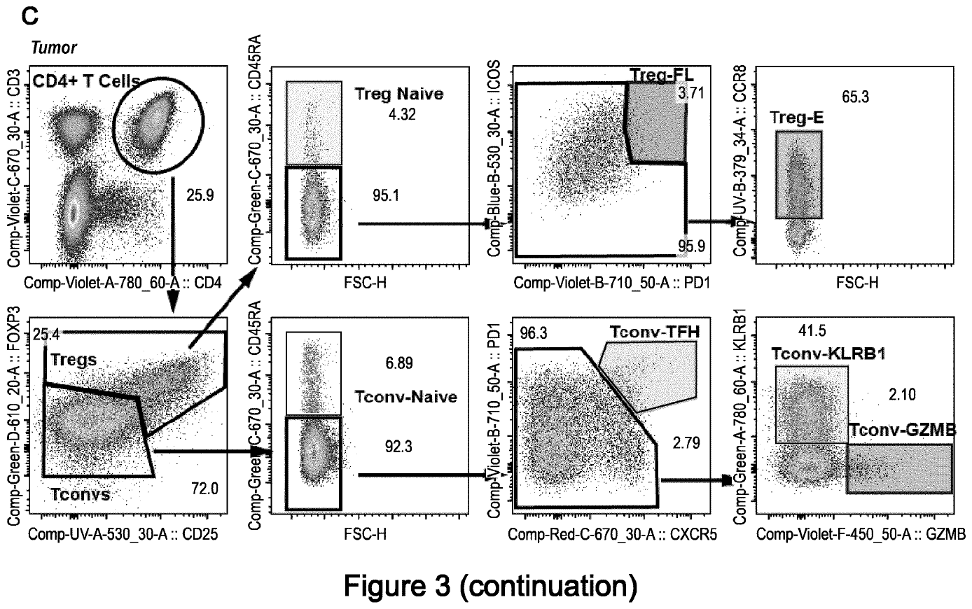

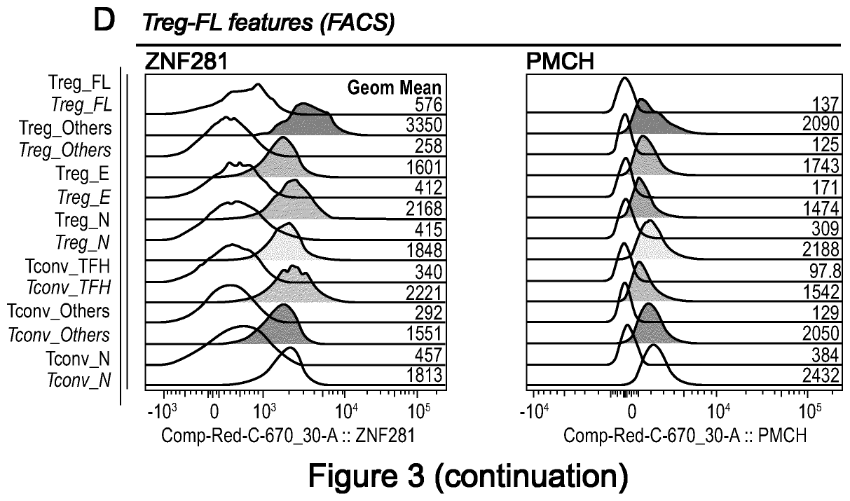

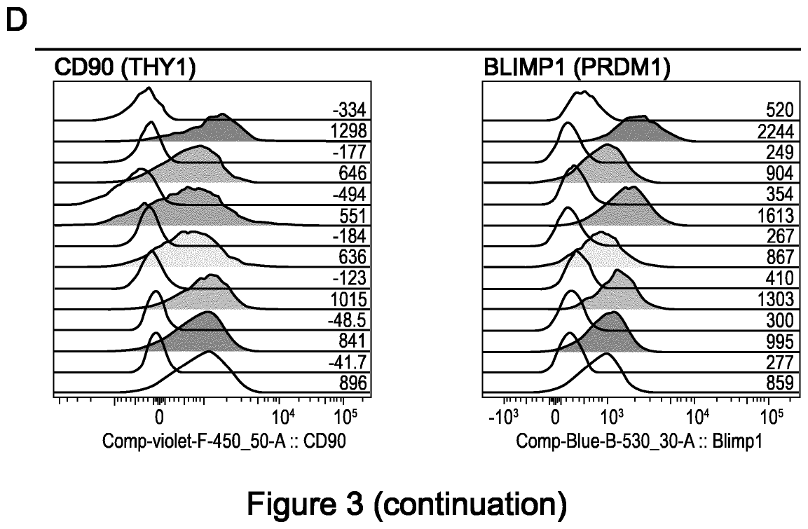

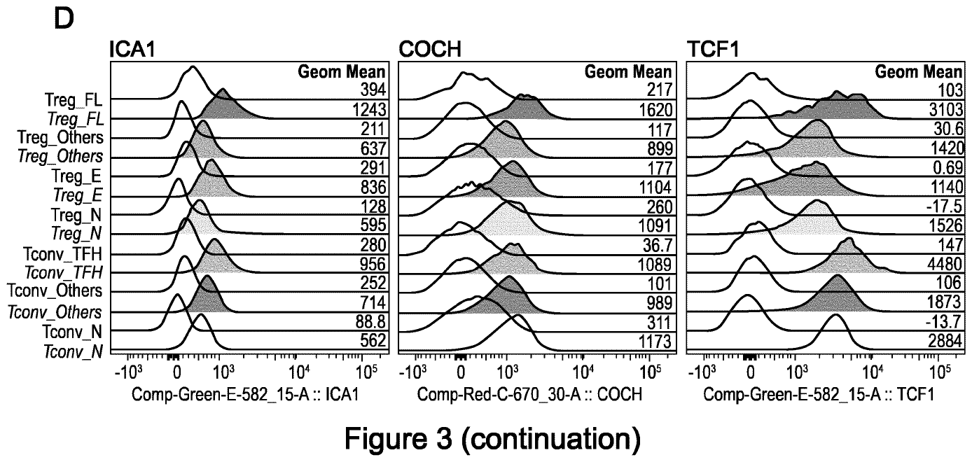

To get more insight about the paths underling cell transitions, RNA velocity trajectory analysis was performed, an alternative unsupervised approach to reconstruct the rate and direction of the developmental patterns of these scRNA-seq data. The velocity vectors highlighted a developmental pathway from naïve to effector cells (both for Tconvs and for Tregs, data not shown) and indicated higher plasticity among Tregs in the LNs and among Tconvs in the tumor. Two additional movements were detected among the tissue imprinted clusters mainly in the LNs, starting from Treg-FL and bifurcating into either Treg-E or Tconv-KLRB1. In a second approach to evaluate the underlying dynamics among these tissue-imprinted cells; Tconv-KLRB1, Treg-FL and Treg-E from the three tissues were pooled, a diffusion map was generated, and the RNA velocity vectors were projected onto this diffusion map (FIG. 3A). The obtained trajectories using this alternative procedure reinforced the identity of Treg-FLs, as precursors of Tconv-TFH or Tconv-KLRB1. A second approach ordering cells across pseudo-time, confirmed these trajectories (data not shown).

In summary, reconstruction of developmental trajectories from transcriptomic patterns revealed multipotent Treg-FL separating into different Treg and Tconv developmental branches.

### TF: Gene Regulatory Programs Explain the Alternative Developmental Pathways of Treg-FL

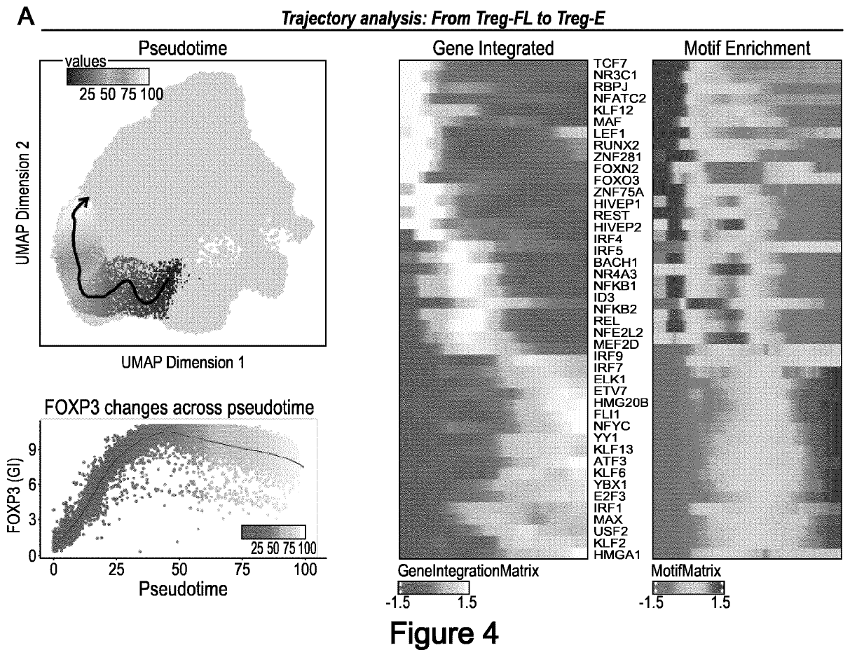

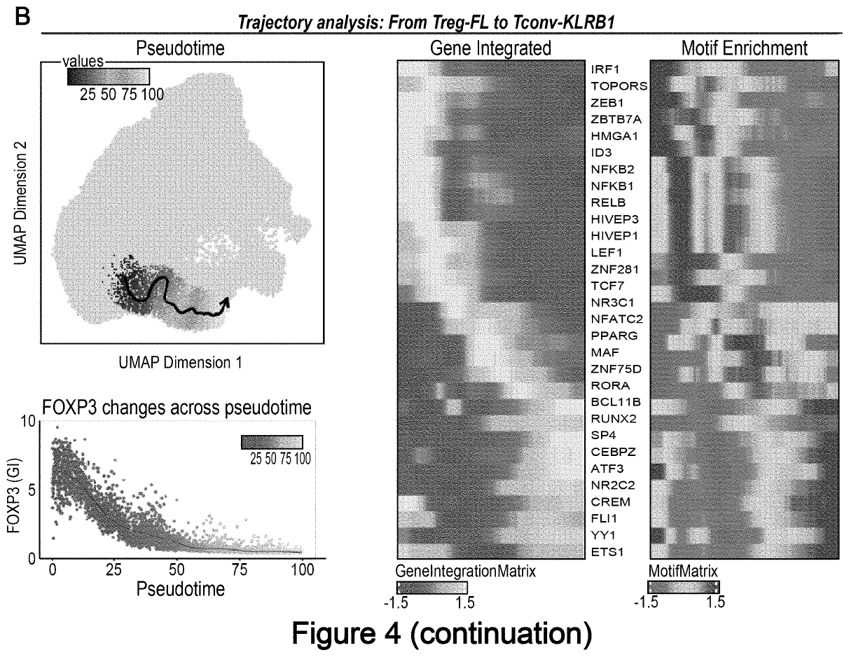

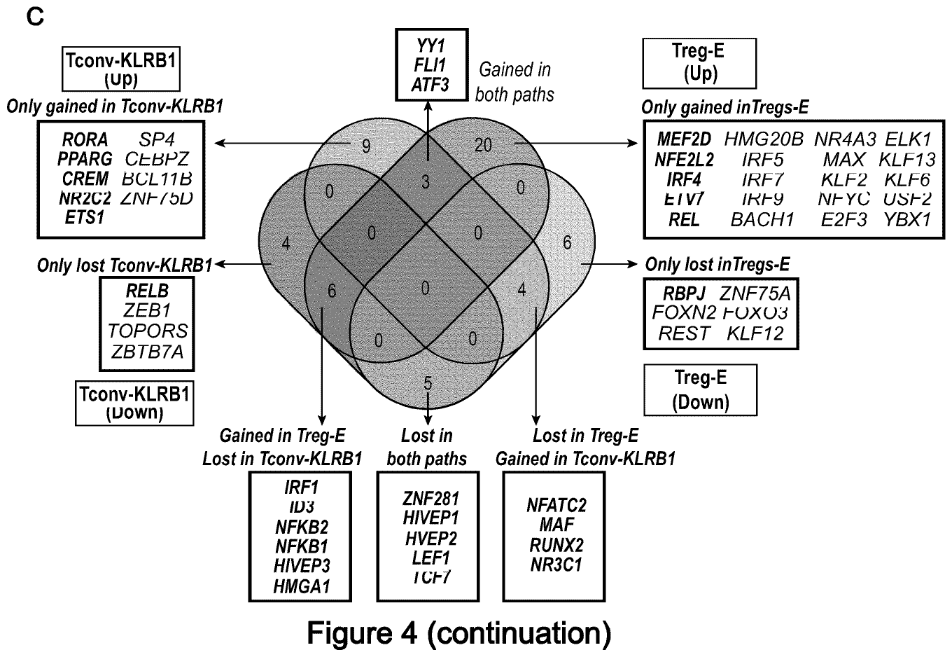

The previous analysis identified Treg-FL as precursors of cells with shared tissue-residency features. To evaluate whether the pluripotency program was also epigenetically defined, the enrichment of selected publicly available ATAC-seq and Chip-seq signatures was quantified. It can be observed that the Treg-FL peaks profile significantly overlaps with multiple epigenetic signatures of precursor cells, including progenitor exhausted CD8+ T cells^{24,25,}progenitor innate lymphocytes²⁶, and the tissue-Treg precursor recently described in the secondary lymphoid organs of mice and committed to the BATF-driven generation of a tissue-residence imprinted progeny¹⁴. Furthermore, Treg-FL and Tconv-TFH shared TFH epigenetic programs and the other tissue-imprinted clusters were enriched in epigenetic signatures matching their identity, i.e., Tconv-GZMB matched with a signature of cytoxic innate-like cells, Tregs-E with tissue-adapted effector Tregs, and Tconv-KLRB1 with tumor-infiltrating TFH and exhausted/dysfunctional cells. Additionally, the DEGs obtained by the integration of scRNA-seq and scATAC-seq were calculated and the newly identified markers were validated by FACS (FIG. 3B-D). Having established the precursor status of Treg-FL, next the TF regulatory program underlying its developmental path towards Treg-E or Tconv-KLRB1 was investigated (FIG. 4A-C). First, trajectory analysis was performed using the scATAC-seq data, which was inferred by pseudotime ordering of TFs for which the motif enrichment in differential peaks positively correlates with the TF expression (gene score and integrated gene expression). Gene integrated and motif enrichment pseudotime heatmaps (FIG. 4A-B), as well as Venn diagrams (FIG. 4C) indicate the dynamics and the distribution of the TFs that drive the transition of Treg-FL towards terminally differentiated regulatory and effector cells. The divergent Treg/Tconv effector program was associated with the alternative activity of ID3, NFKB1-2, HIVEP3, HMGA1, and IRFI (transcriptional regulator of IFNs and IFN-inducible genes involved in Tr1 differentiation²⁷), which are lost in Tconv-KLRB1 and increased in Treg-E clusters. Conversely, NFATC2, MAF, RUNX2 and NR3C1 (repressor of effector T cells during exhaustion²⁸) increase during Tconv-KLRB1 development and decrease in the Treg-E trajectory. Unique to the Treg-FL/Treg-E transition was the increase of NFE2L2, IRF4, REL, ETV7 and MEF2D (transcription regulator controlling suppressive function and IL-10, CTLA-4, and ICOS expression in Treg cells)²⁹. Furthermore, unique to the Treg-FL/Tconv-KLRB1 transition was the increase of PPARG, CREM and RORA (regulators of Th17 gene signature³⁰ ).

The TF: target regulatory networks was then inferred, selecting the TFs that were enriched along the polarization time course and the corresponding regulated genes selected from the DEGs in Treg-E (data not shown)or Tconv-KLRB1 populations (data not shown). The obtained networks uncovered the regulatory programs explaining the expression of genes associated to the Treg and Tconv identity, as well as specific molecular traits and effector function of each cell type. For example, the Treg program explains the expression of key Treg genes, such as FOXP3, CTLA-4, LRRC32 (GARP), and RARA (retinoic acid receptor); while the Tconv-KLRB1 transcriptional network coordinated the expression of BHLHE40, THEMIS, CXCL13, IFNG and IL-21 underlying the antitumoral function of this population (data not shown).

Finally, compatible with the hypothesis of ex-Treg development, the Tconv-KLRB1 pattern of chromatin accessibility at the FOXP3, RORC and IL17A/F loci strongly resembles a published mouse ex-Tregs profile characterized by the acquisition of a Th17-like epigenetic program (data not shown). These data suggest that KLRB1 cells in humans could comprise ex-Treg cells expressing a Th17-like program.

Overall, the combined scRNA-seq and scATAC-seq analysis of tumor samples revealed the transcriptional program underlying the developmental bifurcation of precursor Treg-FLs into tissue imprinted Treg or Tconv cells, which could be used as basis for the design of novel immune modulatory therapies targeting CD4+ T cells.

### Identification of Markers of Migration and Tissue-Residency

The characteristics of the migrating cells (Tregs or Tconvs) versus the resident onesnwere compared for each tissue separately. The cells coming from clones present in both, LN and tumors were taken as migrating cells. the cells from locally expanded clones (clones expanded only in one tissue, without any cell in the other tissue) were taken as resident cells. After, the characteristic of migrating and resident Tregs and Tconvs were explored by tissue. For example: focusing in the LNs, the cells from clones also present in tumor were compared with the clones expanded only in the LNs. As the inventors separated in Tregs and Tconvs, the shared features (those features shared by both migrating Treg/Tconv) and the specific features (those features only displayed by each type of cell) were then explored;

Using this approach unique molecular signatures of Tregs and Tconvs migration and tissue-residency in tumors and lymph nodes were identified as ilustrated below for surface expression and chemokine/integrin encoding genes:
1) a unique molecular signature of migrating Tregs (mTreg) present in the LNs: CCR8, HAVCR2, IL2RB, and LAIR2;
2) a unique molecular signature of migrating Tconvs (mTconvs) present in the LNs: GLUL, HLA-A, HLA-DRA, NKG7 and SEC61G;
3) a unique molecular signature of migrating Tregs (mTreg) present in the Tumor: CCR8, CTLA4, SDC4;
4) a unique molecular signature of migrating Tconvs (mTconvs) present in the tumor: CLEC2B, and HLA-DQA1;
5) a unique molecular signature of resident Tregs (rTreg) present in the LNs: CCR7, LEF1;
6) a unique molecular signature of resident Tconvs (rTconvs) present in the LNs: CCR7, LEF1, SELL, SESN1 and TCF7;
7) a unique molecular signature of resident Tregs (rTreg) present in the Tumor: ICAM3, IL12RB2, IL27RA, LAMP1, LGALS3, TNFRSF14, TNFRSF18, TNFRSF4; and
8) a unique molecular signature of resident Tconvs (rTconvs) present in the tumor: CALM1, CALM3, ICAM3, IL6ST, LGALS3, SIRPG, BTLA, CXCL13.

Then, these unique features can be used to modulate Tregs and Tconvs during cancer.

### Isolation of Precursor Treg-FL for Therapeutic Applications

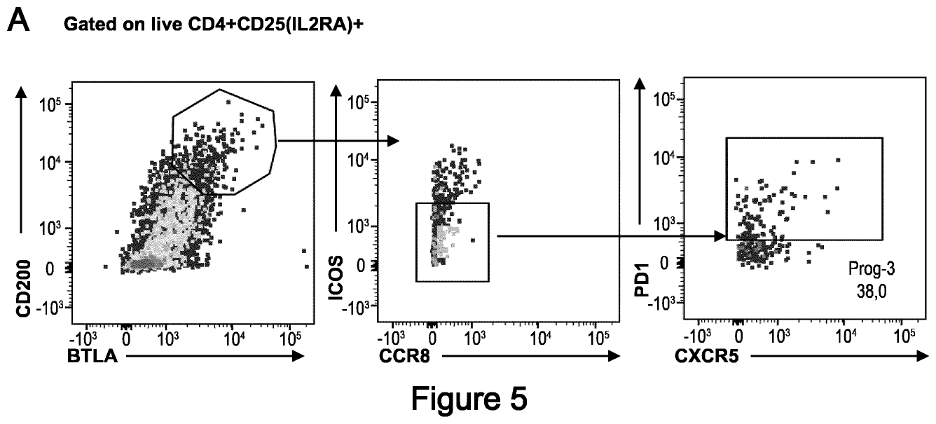

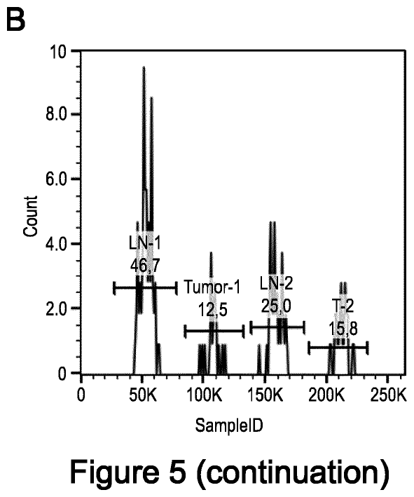

With the aim to properly isolate precursor Treg-FL, differentially expressed and/or accessible genes characterizing Treg-FL and coding surface proteins were validated by FACs and different antibody combinations were tested. The gating strategy shown in FIG. 5A-B efficiently identifies precursor Tregs-FL in lymph nodes and tumors of NSCLC patients allowing their isolation for therapeutic applications.

### IRF1 Protein Precursor Tregs-FL Expression is Upregulated in Peripheral and Tumor-Associated Tregs and its Deletion Impacts Tumor Treg Accumulation and Function

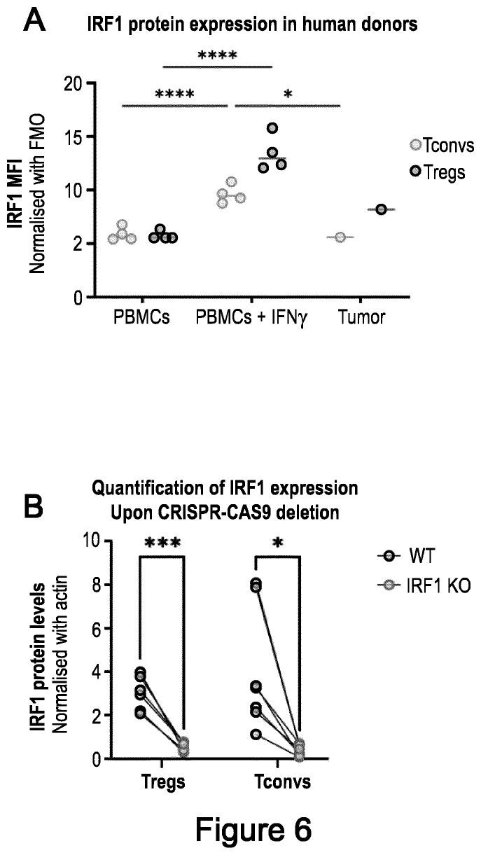

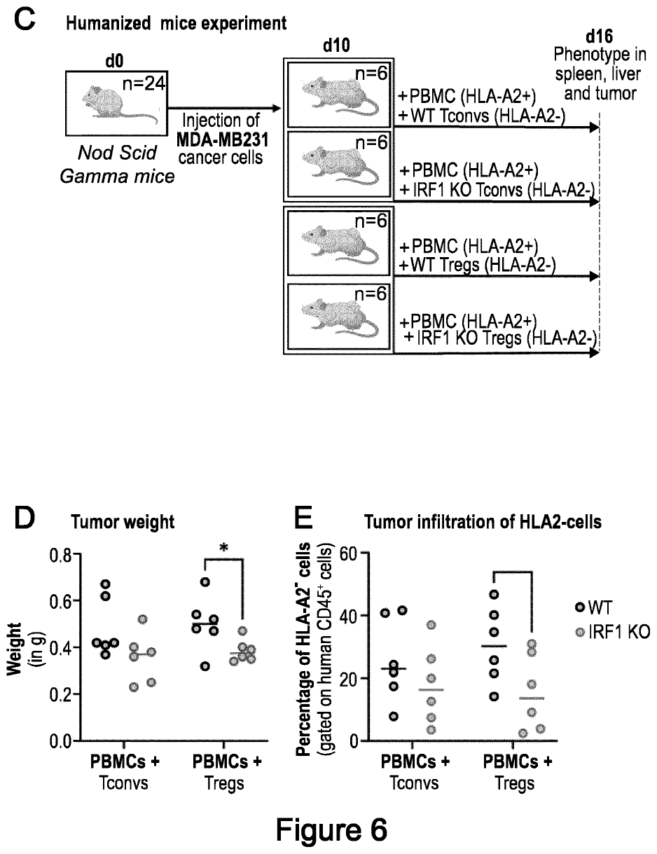

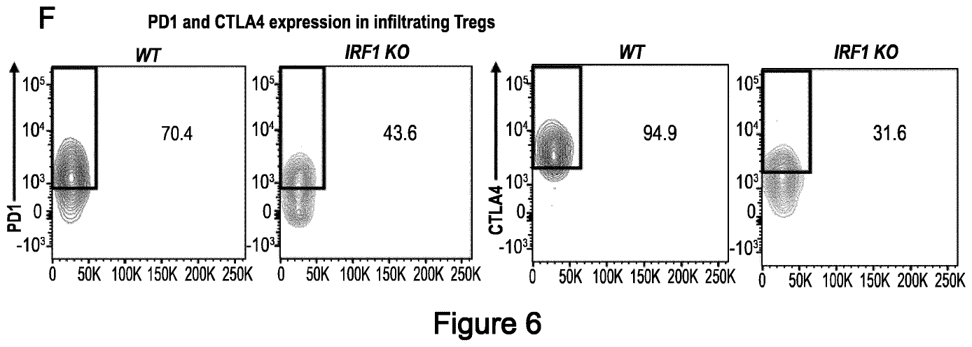

To provide a proof of concept of the therapeutic potential of the molecules highlighted by the previous analysis, IRF1 was selected as a candidate to destabilize resident Tregs. IRF1 was identified as upregulated in the transition from precursor Treg-FL to resident Tres, thus IRF1Treg expression and the effect of its deletion were evaluated. Tumor resident Tregs from a breast cancer patient expressed 1.5 more IRF1 protein compared to the Tconv counterparts, as suggested by the scRNA-seq data (FIG. 6A). Moreover, as IRF1 expression is known to be induced by IFNγ, and as IFNγ is highly present in the tumor microenvironment, we stimulated HD PBMCs with 1000 IU/mL of human recombinant IFNγ overnight, and then evaluated IRF1 expression. Blood Tregs and Tconvs expressed similar levels of IRF1. However, IFNγ stimulation increased Treg IRF1 expression by a factor 2.5 while only by a factor 1.8 for Tconvs, compared to unstimulated condition (FIG. 6A).

To understand the biological role of IRF1 on CD4+ Tregs and Tconvs, Tregs and Tconvs that were genetically knock-out for this gene, using the clustered regularly interspaced short palindromic repeats (CRISPR)-associated protein 9 (CRISPR-Cas9) technology (FIG. 6A). Briefly, Tregs and Tconvs were FACS-sorted at high purity as CD4+CD127-CD25hi cells from CD25 enriched PBMCs and CD4+CD127+CD25low cells from CD4 enriched PBMCs, respectively. After a short in vitro expansion with activation beads, cells were electroporated either with a CRISPR-Cas9 ribonucleoprotein (crRNP) containing a duplex of IRF1-targeting RNAs (IRF1KO), or with an empty crRNP (control, WT). KO efficacy was monitored using western blot (WB) and flow cytometry. As illustrated in FIG. 6B, using this approach, 80% to 95% of Tregs and Tconvs lost IRF1 protein expression 4 days upon electroporation (FIG. 6B).

To investigate whether in the tumor context-where the downregulation of IRF1 in Tconvs and its upregulation in Tregs was originally observed-the absence of IRF1 could impact human Tconv and Treg function, a xenograft model was used. This model consists in the implantation of human tumor cells into immunocompromised mice, to avoid reaction of the mouse immune system against the human tumor tissue. Nod Scid Gamma (NSG) mice were subcutaneously grafted with MDA-MB231 triple negative human breast cancer cells. At day 10, when tumors were palpable, mice were co-injected with human PBMCs and with WT or IRF1 KO Tregs or Tconvs. Tumor-bearing mice were sacrificed 6 days after the injection of T cells to analyze their frequencies and phenotype in the spleen, liver, and tumor (FIG. 6C). Tumor weight was measured at day 16, and a lower weight was detected in tumor injected with IRF1 KO Tregs compared to tumor injected with WT Tregs (FIG. 6D). When comparing the infiltration levels of T cells in the three organs, no effect of IRF1 deletion in the infiltration of Tconvs and Tregs in the spleen and the liver was observed (data not shown). However, in the tumor, a tendency of lower proportion of IRF1 KO Tregs compared to WT ones but no effect on Tconvs was detected (FIG. 6E). In addition, no major phenotypic differences were found in the spleen and in the liver, between WT and IRF1-depleted Tconvs and Tregs, except for a reduced surface expression of PD1 in IRF1 KO Tregs (Data not shown). In contrast, in the tumor, IRF1 deletion on Treg impacted their phenotype, with downregulation of PD1 and CTLA4 (FIG. 6F). PD1 and CTLA-4 are immune checkpoint proteins highly expressed in Tregs and essential for their function. Therefore, these results suggests that IRF1 expression in tumor-infiltrated Tregs is required for the maintenance of Treg phenotype and function. Taken together, these results indicate that IRF1 may influence the accumulation and phenotype of Tregs in tumors.

### DISCUSSION

The advent of cancer immunotherapy has renewed interest in understanding anti-tumor T cell responses, but little is known about the diversity of CD4+ T cells in the LNs and its relationship with the tumor microenvironment^{3,10,31}. By integrating scRNA-seq, scTCR-seq and scATAC-seq profiling of CD4+ Tregs and Tconvs from NSCLC patients, an unprecedented level of granularity was achieved, facilitated by three key methodological aspects. First, equal numbers of Tregs and Tconvs were admixed which allowed a superior resolution for characterizing Treg subpopulations. Second, the inclusion of patient-matched tumor, LNs, and blood specimens allowed tracking clone fates across tissues and clusters. And third, the combination of single-cell omics facilitated the reconstruction of TF: target networks underlying the dynamics of CD4+ T responses.

This approach revealed a tissue residency program driven by BATF, MAF and IRF4 as a hallmark of CD4+ T cell precursors, and more differentiated effectors and regulators enriched in tumor specificities³²⁻³⁴. Analysis of expansion/migration patterns at the subpopulation level, indicated that Treg-E, and Tconv-GZMB,-TFH and -KLRB likely recognize their cognate antigen in LNs, where they expand, and then migrate into the tumors. Interestingly, this group of self-sustaining cells share a follicular program, and may coordinate the B-and T-cell antitumoral responses in the LNs and tertiary lymphoid structure or TLS³⁵. Moreover, developmental pathways reconstruction indicated that Treg-FL cells could act as precursors of Treg-E, or alternatively differentiate into Tconv-KLRB1 cells with a Th17-like phenotype.

Tissue-imprinted CD4+ T cells have been recently identified as key players in tumor specific responses, but their localization in the LNs and information on their biology remain poorly addressed. Feuerer's team has identified a TFH-like differentiation program in Tregs guided by BATF⁹, associated with tumor residency and tissue repair functions. These results support these observations at the transcriptomic and epigenetic level and extend them beyond Treg-E to a set of interrelated CD4+ T cell subpopulations including Treg-FL, Tconv-GZMB, −TFH and −KLRB1. Additionally, the inventors uncovered that Treg-FL are precursors of both tissue-imprinted Treg-E and Tconv-KLRB1. The progenitor properties of follicular-like T cells have been recently identified by several teams. Initially, these stem cell-like characteristics were attributed to follicular-like CD8+ T cells and more recently to CD4+ TFH T cells³⁶⁻³⁹. Along the same lines, a murine tissue-Treg precursor residing in LNs has been recently identified, which highly resembles but is nevertheless different from the Treg-FL precursors here described in humans¹⁴. Notably, the follicular-like phenotype of T cells has been associated to response to immune checkpoint blockers. CD8+ CXCR5+ circulating T cells have been identified as a key population responding to a-PD1 in tumor mouse models. Subsequent studies described TLS formation and CXCL13 as a biomarker of response to a-PD1 therapy in mouse and humans^{40,41}, which echoes the tissue-imprinted T cells described here.

Several therapeutic implications emerge from these results. On one side, CD4+ T cells in the LNs share expanded clonotypes with tumor CD4+ T cells, underlying the accumulation of tumor-specific cells, which show less signs of exhaustion compared to their tumor-infiltrating counterparts and thus represent promising targets for immune modulation⁴². On the other side, LNs can fuel the tumor not only with anti-tumoral cells but also with suppressive Treg cells³. On mature reflection, reprogramming the T cells in the LNs to disable immunosuppressive cells, and/or avoiding LN surgical removal before immunotherapy treatment, should facilitate the therapeutic induction of potent anti-tumor T cell responses. Along these lines, the transcriptional regulatory networks and transcription factors described here (as IRF1) could guide the therapeutic manipulation of the developmental pathways that induce Treg destabilization or force the path to the induction of potent CD4+ T cell effectors. Furthermore, the tissue imprinting program identified by the present study could be used to manipulate CAR-T cells to improve their recruitment to and retention in the tumor⁴³. Overall, this work provides a comprehensive transcriptomic and epigenetic characterization of CD4+ T cells in NSCLC and provides novel cues and insights for future immunotherapy development targeting CD4+ T cells in cancer.

### REFERENCES

1. Lee, J.-U., Kim, L.-K. & Choi, J.-M. Revisiting the Concept of Targeting NFAT to Control T Cell Immunity and Autoimmune Diseases. Front. Immunol. 9, 2747(2018 ).
2. Wing, J. B., Tanaka, A. & Sakaguchi, S. Human FOXP 3+ Regulatory T Cell Heterogeneity and Function in Autoimmunity and Cancer. Immunity 50, 302-316 (2019).
3. du Bois, H., Heim, T. A. & Lund, A. W. Tumor-draining lymph nodes: At the crossroads of metastasis and immunity. Sci. Immunol. 6, eabg3551 (2021).
4. Oh, D. Y. et al. Intratumoral CD4+ T Cells Mediate Anti-tumor Cytotoxicity in Human Bladder Cancer. Cell 181, 1612-1625. e13 (2020).
5. Cohen, M. et al. The interaction of CD 4+ helper T cells with dendritic cells shapes the tumor microenvironment and immune checkpoint blockade response. Nat Cancer 3, 303-317 (2022).
6. Oliveira, G. et al. Landscape of helper and regulatory antitumour CD 4+ T cells in melanoma. Nature 605, 532-538 (2022).
7. Zemmour, D. et al. Single-cell gene expression reveals a landscape of regulatory T cell phenotypes shaped by the TCR. Nat Immunol 19, 291-301 (2018).
8. Miragaia, R. J. et al. Single-Cell Transcriptomics of Regulatory T Cells Reveals Trajectories of Tissue Adaptation. Immunity 50, 493-504. e7 (2019).
9. Delacher, M. et al. Single-cell chromatin accessibility landscape identifies tissue repair program in human regulatory T cells. Immunity 54, 702-720. e17 (2021).
10. Boari, J. T. et al. Tumor invasion in draining lymph nodes is associated with Treg accumulation in breast cancer patients. Nature Communications (2020) doi: 10.1038/s41467-020-17046-2.
11. Zhao, X., Shan, Q. & Xue, H.-H. TCF1 in T cell immunity: a broadened frontier. Nat Rev Immunol (2021) doi: 10.1038/s41577-021-00563-6.
12. Christie, D. & Zhu, J. Transcriptional Regulatory Networks for CD 4 T Cell Differentiation. in Transcriptional Control of Lineage Differentiation in Immune Cells (eds. Ellmeier, W. & Taniuchi, I.) vol. 381 125-172 (Springer International Publishing, 2014).

13. Milner, J. J. & Goldrath, A. W. Transcriptional programming of tissue-resident memory CD8+ T cells. Current Opinion in Immunology 51, 162-169 (2018). 14. Delacher, M. et al. Precursors for Nonlymphoid-Tissue Treg Cells Reside in Secondary Lymphoid Organs and Are Programmed by the Transcription Factor BATF. Immunity 52, 295-312. e11 (2020).

15. Akbaba, H. Resident Memory T Cells. in Cells of the Immune System (eds. Fuchs, O. & Shamsadin Athari, S.) (IntechOpen, 2020). doi: 10.5772/intechopen.90334.
16. Thakore, P. I. et al. The chromatin landscape of Th17 cells reveals mechanisms of diversification of regulatory and pro-inflammatory states. http://biorxiv.org/lookup/doi/10.1101/2022.02.26.482041 (2022) doi:10.1101/2022.02.26.482041.
17. Lowery, F. J. et al. Molecular signatures of antitumor neoantigen-reactive T cells from metastatic human cancers. Science 375, 877-884 (2022).
18. Gueguen, P. et al. Contribution of resident and circulating precursors to tumor-infiltrating CD8⁺ T cell populations in lung cancer. Sci. Immunol. 6, eabd5778 (2021).
19. Wein, A. N. et al. CXCR6 regulates localization of tissue-resident memory CD8 T cells to the airways. Journal of Experimental Medicine 216, 2748-2762 (2019).
20. Shyer, J. A., Flavell, R. A. & Bailis, W. Metabolic signaling in T cells. Cell Res 30, 649-659 (2020).
21. Plitas, G. et al. Regulatory T Cells Exhibit Distinct Features in Human Breast Cancer. Immunity 45, 1122-1134 (2016).
22. Sivasankar, B. et al. CD59 Blockade Enhances Antigen-Specific CD4⁺ T Cell Responses in Humans: A New Target for Cancer Immunotherapy? J Immunol 182, 5203-5207 (2009).
23. Miyara, M. et al. Functional Delineation and Differentiation Dynamics of Human CD4+ T Cells Expressing the FoxP3 Transcription Factor. Immunity 30, 899-911 (2009).
24. Miller, B. C. et al. Subsets of exhausted CD 8+ T cells differentially mediate tumor control and respond to checkpoint blockade. Nat Immunol 20, 326-336 (2019).
25. Pritykin, Y. et al. A unified atlas of CD 8 T cell dysfunctional states in cancer and infection. Molecular Cell 81, 2477-2493. e10 (2021).
26. Shih, H.-Y. et al. Developmental Acquisition of Regulomes Underlies Innate Lymphoid Cell Functionality. Cell 165, 1120-1133 (2016).
27. Karwacz, K. et al. Critical role of IRF 1 and BATF in forming chromatin landscape during type 1 regulatory cell differentiation. Nat Immunol 18, 412-421 (2017).
28. Acharya, N. et al. Endogenous Glucocorticoid Signaling Regulates CD 8+ T Cell Differentiation and Development of Dysfunction in the Tumor Microenvironment. Immunity 53, 658-671.e6 (2020).
29. Di Giorgio, E. et al. MEF2D sustains activation of effector Foxp 3+ Tregs during transplant survival and anticancer immunity. Journal of Clinical Investigation 130, 6242-6260 (2020).
30. Lee, J.-Y. et al. RORa enforces stability of the T-helper-17 cell effector program. http://biorxiv.org/lookup/doi/10.1101/2020.12.15.422921 (2020) doi: 10.1101/2020.12.15.422921.
31. Alonso, R. et al. Induction of anergic or regulatory tumor-specific CD 4+ T cells in the tumor-draining lymph node. Nat Commun 9, 2113 (2018).
32. Alvisi, G. et al. IRF4 instructs effector Treg differentiation and immune suppression in human cancer. Journal of Clinical Investigation 130, 3137-3150 (2020).
33. Seo, H. et al. BATF and IRF4 cooperate to counter exhaustion in tumor-infiltrating CAR T cells. Nat Immunol 22, 983-995 (2021).
34. Vasanthakumar, A. et al. The transcriptional regulators IRF4, BATF and IL- 33 orchestrate development and maintenance of adipose tissue-resident regulatory T cells. Nat Immunol 16, 276-285 (2015).
35. Gu-Trantien, C. et al. CXCL13-producing TFH cells link immune suppression and adaptive memory in human breast cancer. JCI Insight 2, e91487 (2017).
36. Jadhav, R. R. et al. Epigenetic signature of PD-1+ TCF1+ CD8 T cells that act as resource cells during chronic viral infection and respond to PD-1 blockade. Proc Natl Acad Sci USA 116, 14113-14118 (2019).
37. Galletti, G. et al. Two subsets of stem-like CD 8+ memory T cell progenitors with distinct fate commitments in humans. Nat Immunol 21, 1552-1562 (2020).
38. Kunicki, M. A., Amaya Hernandez, L. C., Davis, K. L., Bacchetta, R. & Roncarolo, M.-G. Identity and Diversity of Human Peripheral Th and T Regulatory Cells Defined by Single-Cell Mass Cytometry. J. I. 200, 336-346 (2018).
39. Xia, Y. et al. BCL6-dependent TCF-1+ progenitor cells maintain effector and helper CD4+ T cell responses to persistent antigen. Immunity 55, 1200-1215. e6 (2022).
40. Hsieh, C.-H. et al. Potential Role of CXCL13/CXCR5 Signaling in Immune Checkpoint Inhibitor Treatment in Cancer. Cancers 14, 294 (2022).
41. Liu, B. et al. Temporal single-cell tracing reveals clonal revival and expansion of precursor exhausted T cells during anti-PD-1 therapy in lung cancer. Nat Cancer 3, 108-121 (2022).
42. Spitzer, M. H. et al. Systemic Immunity Is Required for Effective Cancer Immunotherapy. Cell 168, 487-502. e15 (2017).
43. Melenhorst, J. J. et al. Decade-long leukaemia remissions with persistence of CD 4+ CAR T cells. Nature 602, 503-509 (2022).
44. Butler, A., Hoffman, P., Smibert, P., Papalexi, E. & Satija, R. Integrating single-cell transcriptomic data across different conditions, technologies, and species. Nat Biotechnol 36, 411-420 (2018).
45. Zappia, L. & Oshlack, A. Clustering trees: a visualization for evaluating clusterings at multiple resolutions. GigaScience 7, (2018).
46. Finak, G. et al. MAST: a flexible statistical framework for assessing transcriptional changes and characterizing heterogeneity in single-cell RNA sequencing data. Genome Biol 16, 278 (2015).
47. La Manno, G. et al. RNA velocity of single cells. Nature 560, 494-498 (2018).
48. Bergen, V., Lange, M., Peidli, S., Wolf, F. A. & Theis, F. J. Generalizing RNA velocity to transient cell states through dynamical modeling. Nat Biotechnol 38, 1408-1414 (2020).
49. van der Geest, K. S. M. et al. Quantifying Distribution of Flow Cytometric TCR-VB Usage with Economic Statistics. PLoS ONE 10, e0125373 (2015).
50. Granja, J. M. et al. ArchR is a scalable software package for integrative single-cell chromatin accessibility analysis. Nat Genet 53, 403-411 (2021).
51. Kuhn, R. M., Haussler, D. & Kent, W. J. The UCSC genome browser and associated tools. Briefings in Bioinformatics 14, 144-161 (2013).

1. A human T cell precursor having a phenotype characterized by: expression of the markers CD3 and CD4; expression of the marker Forkhead Box P3 (FOXP3) and/or high expression of the marker Interleukin 2 Receptor Subunit Alpha (IL2RA); expression or high expression of the marker Inducible T Cell Costimulator (ICOS); and high expression of the marker Programmed Cell Death 1 (PD1).

2. The T cell precursor according to claim 1, which further comprises high expression of at least one marker selected from the group consisting of: Cytotoxic T-Lymphocyte Associated Protein 4 (CTLA-4), Basic Leucine Zipper ATF-Like Transcription Factor (BATF), Islet Cell Autoantigen 1 (ICA1), Cochlin (COCH), Pro-Melanin Concentrating Hormone (PMCH), Zinc Finger Protein 281 (ZNF281), Thy-1 Cell Surface Antigen (THY.1), CD200, Interferon Regulatory Factor 4 (IRF4), T Cell Immunoreceptor With Ig And ITIM Domains (TIGIT), Thymocyte Selection Associated High Mobility Group Box (TOX), B-Lymphocyte-Induced Maturation Protein 1 (PRDM1), C-X-C Motif Chemokine Ligand 13 (CXCL13), B-and T-lymphocyte attenuator (BTLA), Insulin-like growth factor 1 receptor (CD221), Glucocorticoid-induced TNFR-related protein (TNFRSF18), Integrin Subunit Alpha 4 (ITGA4), Interleukin 21 Receptor (IL21R), leucine rich repeat containing 32 (LRRC32), Rhotekin 2 (RTKN2), IKAROS Family Zinc Finger 2 (IKZF2), and CD151.

3. The T cell precursor according to claim 1, which comprises expression of the markers CD3, CD4 and ICOS; and high expression of the markers IL2RA, CD200, BTLA and PD1.

4. The T cell precursor according to claim 1, which is a precursor of both regulatory T cells (Tregs) and effector conventional T cells (Tconvs).

5. The T cell precursor according to claim 1, wherein said T cell precursor or derived regulatory T cells or conventional T cells express tissue-imprinting markers.

6. The T cell precursor according to claim 5, wherein the derived regulatory T cells or conventional T cells further express at least one transcription factor of T cell activation and differentiation chosen from Jun Proto-Oncogene, AP-1 Transcription Factor Subunit (JUN), RELB Proto-Oncogene, NF-KB Subunit (RELB), Fos Proto-Oncogene, AP-1 Transcription Factor Subunit (FOS), Interferon regulatory factor 4 (IRF4), and Basic Leucine Zipper ATF-Like Transcription Factor (BATF); are mainly found in both lymph nodes and tumors, but not in blood; and are enriched in tumor reactivity.

7. The T cell precursor according to claim 1, which is isolated from blood, tonsil, spleen, bone marrow, lymph node or tumor; which is enriched in lymph node and tumor compared to blood; and/or which is produced from induced pluripotent stem cells (iPS).

8. A method of differentiation of the precursor cell according to claim 1 into regulatory T cells, comprising inducing the expression of a transcription factor program comprising upregulation of at least one of MEF2D, NFE2L2, IRF4, ETV7, REL, HMG20B, IRF5, IRF7, IRF9, BACH1, NR4A3, MAX, KLF2, NFYC, E2F3, ELK1, KLF13, KLF6, USF2, YBX1, IRF1, ID3, NFKB2, NFKB1, HIVEP3, HMGA1, YY1, FLI1 and ATF3; and downregulation of at least one of RBPJ, FOXN2, REST, ZNF75A, FOXO3, KLF12, NFATC2, MAF, RUNX2, NR3C1, ZNF281, HIVEP1, HIVEP2, LEF1 and TCF7 in the cell.

9. A method of differentiation of the precursor cell according to claim 1 into conventional T cells, comprising inducing the expression of a transcription factor program comprising upregulation of at least one of RORA, PPARG, CREM, NR2C2, ETS1, SP4, CEBPZ, BCL11B, ZNF75D, NFATC2, MAF, RUNX2, NR3C1, YY1, FLI1 and ATF3; and downregulation of at least one of RELB, ZEB1, TOPORS, ZBTB7A, IRF1, ID3, NFKB2, NFKB1, HIVEP3, HMGA1, ZNF281, HIVEP1, HIVEP2, LEF1 and TCF7 in the cell.

10. The method according to claim 8, wherein expression of the transcription factor program is induced using modulator(s) of the transcription factor(s) expression or activity selected from the group consisting of: small organic molecules, antibodies, peptides, aptamers, interfering RNA molecules, antisense nucleic acids, ribozymes, genome and epigenome editing complexes, dominant negative mutants, protein fragments and other agonists or antagonists; and/or which further comprises the expansion of the derived regulatory T cells or conventional T cells.

11. A modified immune cell obtained from the T cell precursor according to claim 1, wherein the modified immune cell is genetically engineered to express a chimeric antigen receptor or exogenous TCR specific for a target antigen.

12. A CAR-T cell or TCR-T cell which is modified to stimulate expression of at least one marker of tissue residency chosen from C-X-C Motif Chemokine Receptor 3 (CXCR3), C-X-C Motif Chemokine Receptor 6 (CXCR6), CD69, Interleukin 1 Receptor Like 1 (IL-1RL1), B-Lymphocyte-Induced Maturation Protein 1 (PRDM1), Ectonucleoside Triphosphate Diphosphohydrolase 1 (ENTPD1 or CD39), CD80, TNF Receptor Superfamily Member 4 (TNFRSF4 or OX40), T-Box Transcription Factor 21 (TBX21 or T-bet), CD38, CD274, ICOS, GITR, and TIGIT and/or inhibit expression of at least one marker of blood circulation chosen from C-C Motif Chemokine Receptor 7 (CCR7), Selectin L (SELL), Sphingosine-1-phosphate receptor 1 (S1PR1) and Krüppel-like Factor 2 (KLF2).

13. A TCR-T cell which comprises an engineered TCR from regulatory T cells or conventional T cells as defined in claim 6.

14. A modulator of a molecular signature of migrating or resident regulatory T cells or conventional T cells in the tumor or lymph node for use in the treatment of cancer to modulate immune infiltration in a tumor by favoring the tissue homing and/or migration in the tumor of effector conventional T cells or impairing the tissue homing and/or migration in the tumor of regulatory T cells, wherein the molecular signature is selected from the group consisting of:
a signature of migrating regulatory T cells Tregs present in the lymph node comprising the expression of CCR8, HAVCR2, IL2RB, and LAIR2 genes;
a signature of migrating conventional T cells present in the the lymph node comprising the expression of GLUL, HLA-A, HLA-DRA, NKG7 and SEC61G genes;
a signature of migrating regulatory T cells present in the Tumor comprising the expression of: CCR8, CTLA4 and SDC4 genes;
a signature of migrating conventional T cells present in the tumor comprising the expression of: CLEC2B, and HLA-DQA1 genes;
a signature of resident regulatory T cells present in the lymph node comprising the expression of: CCR7 and LEF1 genes;
a signature of resident conventional T cells present in the lymph node are comprising the expression of: CCR7, LEF1, SELL, SESN1 and TCF7 genes;
a signature of resident regulatory T cells present in the Tumor comprising the expression of: ICAM3, IL12RB2, IL27RA, LAMP1, LGALS3, TNFRSF14, TNFRSF18 and TNFRSF4 genes; and
a signature of resident conventional T cells present in the tumor comprising the expression of: CALM1, CALM3, ICAM3, IL6ST, LGALS3, SIRPG, BTLA and CXCL13 genes.

15. A pharmaceutical composition comprising a therapeutically effective amount of T cell precursor according to claim 1.

16. A method of treating cancer, acute or chronic inflammatory diseases, autoinflammatory diseases, miscarriage, allergic diseases, autoimmune or infectious diseases, graft-versus-host disease and graft-rejection, and tissue repair in a human subject comprising administering the pharmaceutical composition of claim 15 to the human subject.
